(19) United States
(12) Patent Application Publication
(10) Pub. No.: US 2023/0149688 A1
(43) Pub. Date: May 18, 2023

(54) Method And System For Selectively Coupling A Blood Collection Pressure Apparatus

(71) Applicant: Terumo BCT, Inc., Lakewood, CO (US)

(72) Inventors: Jeremy PARSONS, Denver, CO (US); Jesse JANZEN, Broomfield, CO (US)

(21) Appl. No.: 17/975,180

(22) Filed: Oct. 27, 2022

Related U.S. Application Data
(60) Provisional application No. 63/280,049, filed on Nov. 16, 2021.

(51) Int. Cl. A61M 39/10; A61M 39/12; A61M 39/26

(52) U.S. Cl. CPC A61M 39/1011; A61M 39/12; A61M 39/26; A61M 2039/1027; A61M 2039/267

(57) ABSTRACT

A selective coupling of a bladder and a bucket of a blood separation system includes mating an integrated connector of the bladder with a receiver disposed in the bucket without disassembling the bucket from the blood separation system. The receiver includes a locking receptacle and is mounted to a portion of the bucket. The integrated connector of the bladder selectively engages with the receiver. When engaged, the connector is locked to the receiver via a locking latch. To disengage the bladder from the bucket, a technician releases the locking latch and removes the bladder from the bucket without disassembling the bucket or the bladder. The engagement and disengagement is performed with a quick interconnect operation. The integrated connector and the receiver are valveless and provide an unimpeded flow path from a fluid source to the bladder.

### CROSS-REFERENCE TO RELATED APPLICATIONS

This application claims the benefit of U.S. provisional application No. 63/280,049, filed on Nov. 16, 2021. The entire disclosure of the above application is incorporated herein by reference.

### BACKGROUND

This section provides background information related to the present disclosure which is not necessarily prior art.

The present disclosure is generally directed to blood separation systems, in particular, toward the attachment of pressure bladders to centrifuge buckets in blood separation systems.

Blood collection and processing play important roles in the worldwide health care system. In conventional large scale blood collection, blood is removed from a donor or patient, separated into its various blood components via centrifugation, filtration, or elutriation and stored in sterile containers for future infusion into a patient for therapeutic use. The separated blood components typically include fractions comprising red blood cells, white blood cells, platelets, and plasma. Separation of blood into its components can be performed continuously during collection or can be performed subsequent to collection in batches, particularly with respect to the processing of whole blood samples. Separation of blood into its various components under highly sterile conditions is critical to many therapeutic applications.

### BRIEF SUMMARY

This section provides a general summary of the disclosure, and is not a comprehensive disclosure of its full scope or all of its features.

At least one example embodiment relates to a selective coupling assembly. The selective coupling assembly includes a bucket, a bladder, and a connector. The bucket includes a wall and a receiver. The wall extends from an open end of the bucket to a closed end of the bucket. The wall at least partially defines a cavity between the open end and the closed end. The receiver is attached to the wall. The receiver includes a body and a latch plate. The body defines a receptacle and a receiver lumen extending between a first side of the body and a second side of the body. The receiver lumen has a lumen axis. The latch plate is slidably attached to the body. The latch plate defines an aperture and having an aperture axis parallel the lumen axis. The bladder defines a sealed expandable chamber and a fluid flow port passing from an interior volume of the sealed expandable chamber to an outside of the bladder. The connector is attached to the bladder. The connector is at least partially in the fluid flow port. The connector includes a base and a plug. The plug protrudes from the base. The connector defines a connector lumen in the base and the plug. The connector lumen provides a fluid flow path from the interior volume of the sealed expandable chamber to an outside of the bladder.

In at least one example embodiment, the receiver is at least partially in the wall.

In at least one example embodiment, the plug further includes a recess and a groove. The recess is offset a first distance from the base of the plug. The groove is defined in a periphery of the plug. The groove is offset a second distance from the base of the plug.

In at least one example embodiment, the connector further includes an O-ring at least partially in the groove.

In at least one example embodiment, the connector comprises a plastic material. The connector is attached to the bladder via at least one weld.

In at least one example embodiment, the connector is configured to move between a lock state with the receiver and an unlocked state with the receiver. In the lock state, the bladder is coupled to the bucket. In the unlocked state, the bladder is decoupled from the bucket.

In at least one example embodiment, in the lock state, the plug is disposed at least partially in the receptacle and a portion of the latch plate is in the recess of the plug.

In at least one example embodiment, in the lock state, a fluid flow path is between the interior volume of the sealed expandable chamber and the receiver lumen of the receiver. The fluid flow path is unimpeded by any valve between the bladder and the receiver.

At least one example embodiment relates to a bladder assembly. The bladder assembly includes a flexible material and a connector. The flexible material defines a sealed expandable chamber. The flexible material defines a fluid flow port passing from an interior volume of the sealed expandable chamber to an outside of the sealed expandable chamber. The connector is attached to the flexible material and at least partially in the fluid flow port. The connector includes a base and a plug. The plug protrudes from the base. The connector defines a connector lumen through the base and the plug. The connector lumen provides a fluid flow path from the interior volume of the sealed expandable chamber to an outside of the flexible material.

In at least one example embodiment, the connector is attached to the flexible material via a seal surrounding the fluid flow port and joining a portion of the flexible material to the base of the connector.

At least one example embodiment relates to a method of coupling a bladder to a bucket. The method includes inserting a bladder including an integral connector into a cavity of a bucket. The method further includes aligning the integral connector with a receiver in a wall of the bucket. The method further includes guiding the integral connector into the receiver. The method further includes applying a force to the integral connector to engage a latch of the receiver with a portion of the integral connector and reduce or prevent axial movement of the integral connector relative to the receiver.

In at least one example embodiment, the guiding includes inserting a selective coupling tool into the cavity of the bucket. The guiding further includes moving the selective coupling tool into contact with a portion of the integral connector. The guiding further includes manipulating the selective coupling tool to cause a movement of the integral connector in a direction of the receiver.

In at least one example embodiment, the moving includes positioning the selective coupling tool relative to a top surface of the bucket. The moving further includes aligning an indicium on a shaft of the selective coupling tool with a reference point at the top surface of the bucket.

In at least one example embodiment, the bladder defines a sealed expandable chamber and a fluid flow port passing from an interior volume of the sealed expandable chamber to an outside of the bladder.

In at least one example embodiment, the integrated connector includes a base and a plug. The plug protrudes from the base. A connector lumen passes through the base and the plug. The connector lumen provides a fluid flow path from the interior volume of the sealed expandable chamber to an outside of the bladder.

At least one example embodiment relates to a method of decoupling a bladder from a bucket. The method includes inserting a selective coupling tool at least partially into a cavity of a bucket in a region between the bladder and a wall of the bucket. The method further includes engaging the selective coupling tool with a latch plate of a receiver at least partially in the wall of the bucket. The method further includes moving the selective coupling tool to cause the latch plate to translate from a lock state, to a unlocked state. In the lock state, a portion of the latch plate is engaged with a portion of a connector of the bladder. In the unlocked state, the portion of the latch plate is disengaged with the portion of the connector of the bladder. The method further includes moving the connector away from the receiver to cause the bladder to separate from the bucket.

In at least one example embodiment, the engaging includes aligning a pin of the selective coupling tool with a corresponding hole defined in the latch plate. The engaging further includes inserting at least a portion of the pin into the corresponding hole.

In at least one example embodiment, the aligning includes aligning an indicium on a shaft of the selective coupling tool with a reference point at a top surface of the bucket.

In at least one example embodiment, the engaging includes contacting the selective coupling tool with a flange of the latch plate.

At least one example embodiment relates to a selective coupling tool. The selective coupling tool includes a shaft and a forked extension. The shaft extends from a proximal end to a distal end. The forked extension protrudes from the distal end. The forked extension has a first side and a second side opposite the first side. The forked extension includes a cradle and a protrusion. The cradle includes a contact surface between the first side and the second side. The protrusion is on the second side.

In at least one example embodiment, the protrusion is a frustoconical protrusion.

In at least one example embodiment, the protrusion is a plate.

In at least one example embodiment, the selective coupling tool further includes a handle and an indicum. The handle is connected to the proximal end of the shaft. The indicium is on the shaft.

In at least one example embodiment, the indicium is etched into a portion of the shaft. The indicum extends around at least a portion of an outer surface of the shaft.

At least one example embodiment relates to a blood separation apparatus. The blood separation apparatus includes a rotor, a bucket, a receiver, a bladder, and a connector. The bucket is attached to the rotor. The bucket includes a wall and a receiver. The wall extends from an open end of the bucket to a closed end of the bucket. The wall defines a cavity between the open end and the closed end. The receiver is attached to the wall. The receiver includes a body and a latch plate. The body defines a receptacle and a receiver lumen extending between a first side of the body and a second side of the body. The receiver lumen has a lumen axis. The latch plate is slidably attached to the body. The latch plate defines an aperture and having an aperture axis parallel to the lumen axis. The bladder defines a sealed expandable chamber and a fluid flow port extending between an interior volume of the sealed expandable chamber to an outside of the bladder. The connector is attached to the bladder. The connector is at least partially in the fluid flow port. The connector includes a base, a plug, and a connector lumen. The plug protrudes from the base. The connector lumen is defined through the base and the plug. The connector lumen provides a fluid flow path from the interior volume of the sealed expandable chamber to an outside of the bladder.

At least one example embodiment relates to a selective coupling assembly. The selective coupling assembly includes a receiver, a bladder, and a connector. The receiver is at least partially within a wall of a bladder holder. The receiver includes a body and a latch plate. The body defines a receptacle and a first lumen extending from a first side of the body to a second side of the body. The first lumen has a lumen axis. The latch plate is slidably attached to the body. The latch plate defines an aperture and having an aperture axis parallel to the lumen axis. The bladder defines an expandable chamber and a fluid flow port passing from an interior volume of the expandable chamber to an outside of the bladder. The connector is attached to the bladder. The connector is at least partially in the fluid flow port. The connector includes a base and a plug. The plug protrudes from the base, The connector defines a connector lumen through the base and the plug. The connector lumen provides a fluid flow path from the interior volume of the expandable chamber to an outside of the bladder.

In at least one example embodiment, the bladder is configured to be moved from a first position outside of the bladder holder to a second position inside the bladder holder. The receiver is at least partially within the bladder holder.

In at least one example embodiment, the connector is configured to be moved between a lock state with the receiver and an unlocked state with the receiver. In the lock state, the bladder is coupled to the bladder holder. In the unlocked state, the bladder is decoupled from the bladder holder. The connector is configured to be moved between the lock state and the unlocked state from a region inside the bladder holder.

In at least one example embodiment, the connector is configured to be moved between the lock state and the unlocked state without use of a tool.

In at least one example embodiment, the connector is configured to be moved between the lock state and the unlocked state by inserting a tool from an outside of the bladder holder into the region.

In at least one example embodiment, the bladder holder is a bucket of a separation apparatus. The wall extends from an open end of the bucket to a closed end of the bucket.

At least one example embodiment relates to an interconnection assembly. The interconnection assembly includes a receiver and a connector. The receiver includes a body and a latch plate. The body defines a receptacle and a receiver lumen extending between a first side of the body to second side of the body. The receiver has a lumen having a lumen axis. The latch plate is slidably attached to the body. The latch plate defines an aperture having an aperture axis parallel to the lumen axis. The connector includes a base and a plug. The plug protrudes from the base. The connector defines a connector lumen through the base and the plug.

In at least one example embodiment, the interconnection assembly further includes a bladder. The bladder defines an expandable chamber and a fluid flow port passing from an interior volume of the expandable chamber to an outside of the bladder. The connector is operatively attached to the fluid flow port such that the connector lumen provides a fluid flow path from the interior volume of the expandable chamber to an outside of the bladder.

In at least one example embodiment, the plug further includes a recess an a compliant portion. The recess is offset a first distance from the base of the plug. The compliant portion is arranged around a periphery of the plug. The compliant portion is configured to create a seal between the plug and the receptacle. The compliant portion is offset a second distance from the base.

In at least one example embodiment, the compliant portion is an elastically flexible ridge protruding from the plug.

Blood separation systems may be used to automate the process of blood componentization. In at least one example embodiment, this process may be performed by loading a blood bag into a metal bucket that is spun, around a rotation axis, to separate the blood into its components. Once separated, the various components may be expressed out of the bucket by displacing the fluid in the blood bag with a bladder (e.g., inflatable, pneumatic, hydraulic, etc.) that is also inside the bucket (e.g., disposed adjacent to and in contact with an outside of the blood bag). These bladders need to be replaced as part of the routine maintenance scheduled for blood separation systems. Replacing the bladders requires a service technician to completely remove each of the buckets from the blood separation system to access and release a threaded nut that secures the bladders in the buckets. This process is time consuming, complex, and poses inherent risk for a service technician. The process can take as long as several hours to complete. As can be appreciated, maintenance of the system and replacement of the bladders is a costly and frustrating procedure for service technicians.

It is with respect to the above issues and other problems that the embodiments presented herein were contemplated.

In at least some embodiments, the present disclosure describes methods, devices, and systems for selectively coupling (e.g., installing and/or removing, etc.) bladders from buckets without requiring full disassembly of the bucket from the blood separation system. In one example, a receiver (e.g., locking receptacle, etc.) is mounted to a portion of the bucket and a separate connector, that selectively engages with the receiver, is attached to the bladder. When engaged, the connector is locked to the receiver via a locking latch. To disengage the bladder from the bucket, a technician may release the locking latch and remove the bladder from the bucket without disassembling the bucket and/or the bladder. The engagement and disengagement may be performed with a quick interconnect operation that each take less than one minute to complete.

The connector (e.g., engaging plug, etc.) may be attached to the bladder (e.g., inflatable bladder, etc.). The connector may include a base and an engaging plug protruding from the base. The connector may be injection molded and welded (e.g., radio frequency (RF), ultrasonically, etc.), or otherwise affixed (e.g., glued, adhered, melted, fastened, etc.), to the bladder. For example, the base of the connector may be a flat sheet, or planar body, that is attached to a body of the bladder.

The receiver may be installed, or otherwise formed, during production or manufacturing of the bucket and/or the blood separation system. Stated another way, when attached (or alternatively, affixed) to the bucket, the receiver becomes a part of the bucket, or bucket assembly. The receiver of the bucket may include an aperture that is configured to receive at least a portion of a mating connector of a bladder. In at least one example embodiment, the receiver may include a spring-loaded latch and locking latch plate. The receiver may include a receptacle or aperture that is configured to receive at least a portion of the engaging plug of the connector. In at least one example embodiment, the receiver may include a spring-loaded latch having a plate that is arranged perpendicular to an axis of the receptacle. The plate may include an aperture that is similar in size to the aperture of the receiver. The aperture of the plate may be arranged concentrically relative to the aperture of the receiver when the plate is in a release position. In one example, the plate may be arranged such that an edge of the aperture of the plate does not block an area of the aperture of the receiver when in the release position. In any event, the plate may shift relative to the aperture of the receiver when the plate is in a lock position. When shifted, the aperture of the plate may be arranged eccentrically relative to the aperture of the receiver. In the lock position, an edge of the aperture of the plate may block a portion of the area of the aperture of the receiver.

When the engaging plug (of the bladder) is inserted into the receptacle of the receiver (of the bucket), and pushed into a trigger position, the connector may press on a trigger pin that causes the spring-loaded latch and plate inside the receiver to move from the release position to the lock position such that the plate engages with a receiving groove in the connector. The receiving groove may be disposed around a periphery of the engaging plug, or a portion thereof. In one example, the receiving groove may be configured as a recess in a body of the engaging plug. When engaged, at least a portion of the edge of the aperture of the plate may insert into the recess in the body of the engaging plug preventing movement of the engaging plug along the axis of the aperture of the receiver and the axis of the engaging plug.

When disengaging the bladder from the bucket, the engagement process may be reversed. For instance, a technician may unlatch the spring-loaded latch (e.g., by hand, with a tool, etc.), that moves the plate from the lock position to the release position such that the plate is disengaged from the receiving groove in the connector. When configured as a recess in the body of the engaging plug, the disengagement of the plate removes the edge of the aperture of the plate completely from the recess in the body of the engaging plug allowing movement of the engaging plug along the axis of the aperture of the receiver and the axis of the engaging plug. In some examples, the spring-loaded latch may hold in the release position, resetting the trigger pin. While in the release position, the engaging plug may be removed from the receiver and the bladder may be completely separated from the bucket. As can be appreciated, this method, device, and system allows for quick changeover of bladders during maintenance operations, saving time versus the conventional disassembly approaches, and providing a reliable interconnection that can be performed without disassembly. Among other things, the methods, devices, and systems described herein allow for the quick selective coupling (e.g., connection and disconnection, etc.) of a bladder and bucket to be performed within a matter of seconds or minutes (e.g., 1-2 minutes) rather than taking hours (e.g., requiring two or more hours of complicated disassembly) as provided by conventional systems.

In at least one example embodiment, the connector may define a lumen (e.g., fluid channel or conduit) extending through the engaging plug and base. The lumen may be in fluid communication with an internal volume of the bladder. The lumen may be unimpeded (e.g., valveless), providing a clear and unrestricted path (e.g., unrestricted by a sealing valve, or flow-limiting valve) from through the connector. Similarly, the receptacle or aperture of the receiver may form a portion of a hollow channel, or lumen, running through the receiver. This lumen of the receiver may be valveless providing a clear and unrestricted path (e.g., unrestricted by a sealing valve, or flow-limiting valve) through the receiver. In at least one example embodiment, a fluid (e.g., pneumatic, hydraulic, etc., and/or combinations thereof), may be conveyed along a flow path to the lumen of the receiver. When the connector is engaged with the receiver, the fluid may move, via the flow path, through the receiver into the lumen of the connector and into the internal volume of the bladder, or vice versa. When fluid is moved into the bladder, the bladder may increase in size (e.g., inflate, grow, expand, etc.) and when fluid is moved out of the bladder (e.g., via pumping or drawing fluid from the bladder along the flow path, etc.), the bladder may decrease in size (e.g., deflate, shrink, contract, etc.).

Further areas of applicability will become apparent from the description provided herein. The description and specific examples in this summary are intended for purposes of illustration only and are not intended to limit the scope of the present disclosure.

### BRIEF DESCRIPTION OF THE SEVERAL VIEWS OF THE DRAWINGS

The drawings described herein are for illustrative purposes only of selected embodiments and not all possible implementations, and are not intended to limit the scope of the present disclosure.

FIG. 1 is a perspective view of a rotor of a separation apparatus in accordance with at least one embodiment.

FIG. 2 is a schematic view, partly in cross-section along a diametral plane, of the separation apparatus of FIG. 1 in accordance with at least one example embodiment.

FIG. 3 is a top view of the rotor of FIG. 1 in accordance with at least one example embodiment.

FIG. 4 is a schematic view, in cross-section along a radial plane, of a separation cell of the rotor of FIG. 1 in accordance with at least one example embodiment.

FIG. 5 is a schematic view, in cross-section along a radial plane, of another separation cell in accordance with at least one example embodiment.

FIG. 6 is a cross-section view of a separation apparatus including a rotor and single separation cell in accordance with at least one example embodiment.

FIG. 7 is a partial cross-sectional view of the separation apparatus of FIG. 6 in accordance with at least one example embodiment.

FIGS. 8A-8C are partial cross-sectional views of a selective coupling assembly of the separation apparatus of FIG. 6 in accordance with at least one example embodiment. FIG. 8A illustrates the selective coupling assembly in release (or disengaged or unlocked) state. FIG. 8B illustrates the selective coupling assembly in an intermediate (or partially engaged state). FIG. 8C illustrates the selective coupling assembly in a lock state (or engaged).

FIGS. 9A-9D depict a selective coupling tool in accordance with at least one example embodiment. FIG. 9A is a first side perspective view of the selective coupling tool. FIG. 9B is a second side perspective view of the selective coupling tool. FIG. 9C is a first side partial perspective view of the selective coupling tool. FIG. 9D is a second side partial perspective view of the selective coupling tool.

FIG. 10 is a partial elevation view of the separation apparatus of FIG. 6 including the selective coupling assembly in the release state and being guided into toward lock state by the selective coupling tool of FIGS. 9A-9D in accordance with at least one example embodiment.

FIGS. 11A-11B relate the selective coupling assembly of FIG. 6 in the lock state in accordance with at least one example embodiment. FIG. 11A is a perspective view of a connector (shown in phantom) and receiver of the selective coupling assembly. FIG. 11B is a perspective view of the receiver.

FIGS. 12A-12B relate to the selective coupling assembly of FIG. 11A in the lock state prior to being unlocked by the selective coupling tool of FIGS. 9A-9D in accordance with at least one example embodiment. FIG. 12A is a perspective view of the selective coupling assembly in the lock state with the tool engaged with a portion of the receiver. FIG. 12B is a perspective view of the tool engaged with the portion of the receiver in the lock state.

FIG. 13 is a perspective view of the selective coupling assembly of FIG. 12A in the disengaged state with a selective coupling tool of FIGS. 9A-9B engaged with the portion of the receiver in accordance with at least one example embodiment.

FIGS. 14A-14B are cross-sectional views of another selective coupling assembly including an engaging plug and a receiver in accordance with at least one example embodiment. FIG. 14A shows the engaging plug engaged with the receiver. FIG. 14B shows the engaging plug disengaged from the receiver.

FIGS. 15A-15B are cross-sectional views of yet another selective coupling assembly including an engaging plug and a receiver in accordance with at least one example embodiment. FIG. 15A shows the engaging plug engaged with the receiver. FIG. 15B shows the engaging plug disengaged from the receiver.

FIGS. 16A-16B are cross-sectional views of yet another selective coupling assembly including an engaging plug and a receiver in accordance with at least one example embodiment. FIG. 16A shows the engaging plug engaged with the receiver. FIG. 16B shows the engaging plug disengaged from the receiver.

FIGS. 17A-17D illustrate another selective coupling tool in accordance with at least one example embodiment. FIG. 17A is a first side perspective view of the selective coupling tool. FIG. 17B is a second side perspective view of the selective coupling tool. FIG. 17C is a first side partial perspective view of the selective coupling tool. FIG. 17D is a second side partial perspective view of the selective coupling tool.

FIGS. 18A-18B relate to unlocking of a selective coupling assembly using the tool of FIGS. 17A-17D in accordance with at least one example embodiment. FIG. 18A is a perspective view of the selective coupling assembly in a lock state. FIG. 18B is a perspective view of the selective coupling assembly in the lock state prior to being moved to a release state, the tool engaging the selective coupling assembly.

FIGS. 19A-19B are schematic cross-section partial views of another separation apparatus in accordance with at least one example embodiment, the separation apparatus including a channel to facilitate use of a selective coupling tool. FIG. 19A shows the selective coupling assembly with the tool partially in the channel. FIG. 19B shows the selective coupling assembly with the tool in the channel and contacting a receiver of the selective coupling assembly.

FIG. 20 is a flowchart of a method for engaging a bladder with a separation cell of a separation apparatus in accordance with embodiments of the present disclosure; and

FIG. 21 is a flowchart of a method for disengaging a bladder from a separation cell of a separation apparatus in accordance with embodiments of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

At least one embodiment of the present disclosure relates to an apparatus for concurrently (or simultaneously) separating, by centrifugation, discrete volumes of a composite liquid (e.g., blood). The apparatus may comprise a centrifuge adapted to receive a number of bags, with the discrete volumes of a composite liquid contained in each of the separation bags, a component transferring means for transferring at least one separated component from each separation bag into a satellite bag connected thereto, a first balancing means for initially balancing the rotor when the weights of the four separation bags are different, and a second balancing means for balancing the rotor when the weights of the separated components transferred into the satellite bags cause an unbalance of the rotor.

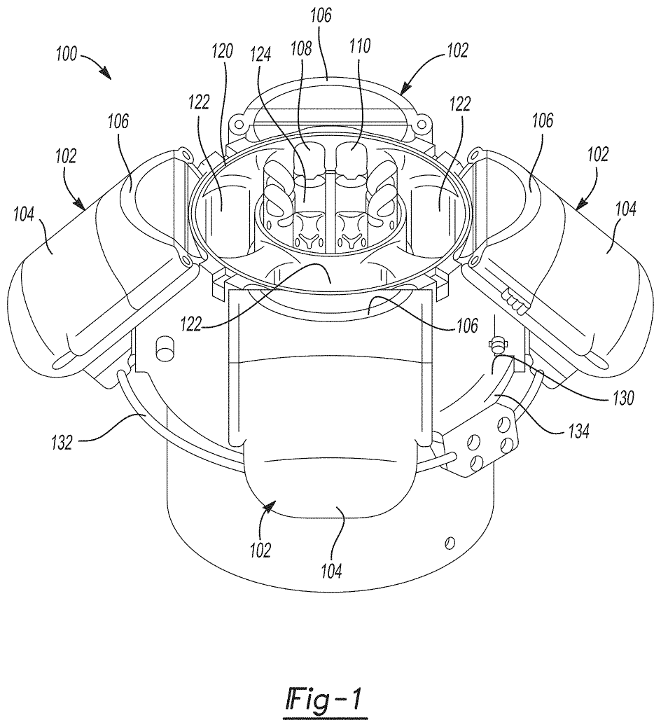

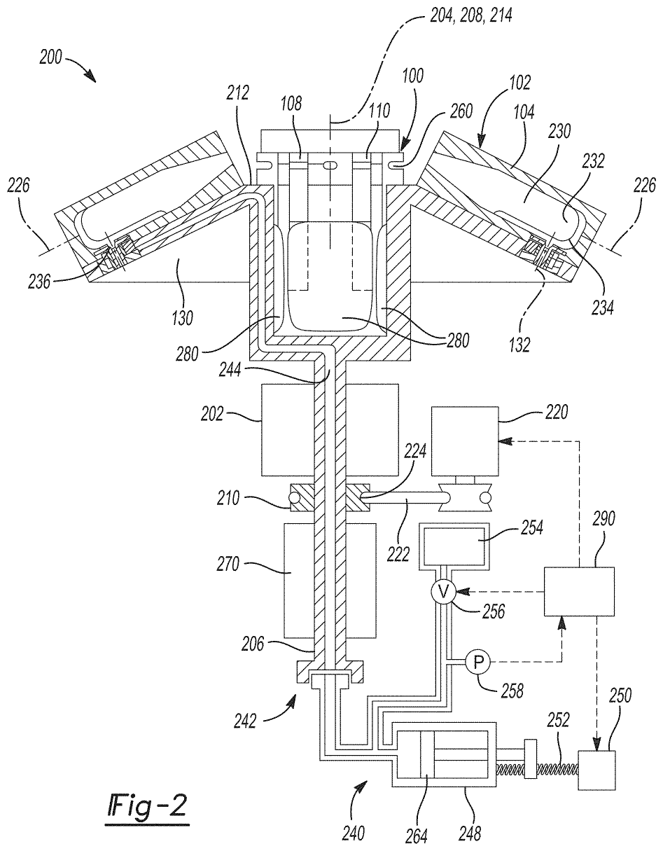

With reference to FIG. 1, rotor 100 of a separation apparatus (see, e.g., centrifuge 200 of FIG. 2) according to at least one example embodiment is provided. The rotor 100 generally includes one or more (e.g., four, as shown) separation cells 102. In at least one example embodiment, the separation cells 102 are identical to one another. Each separation cell 102 includes a container 104 (also referred to herein interchangeably as a “bucket”). Each of the containers 104 may have the general shape of a rectangular parallelepiped.

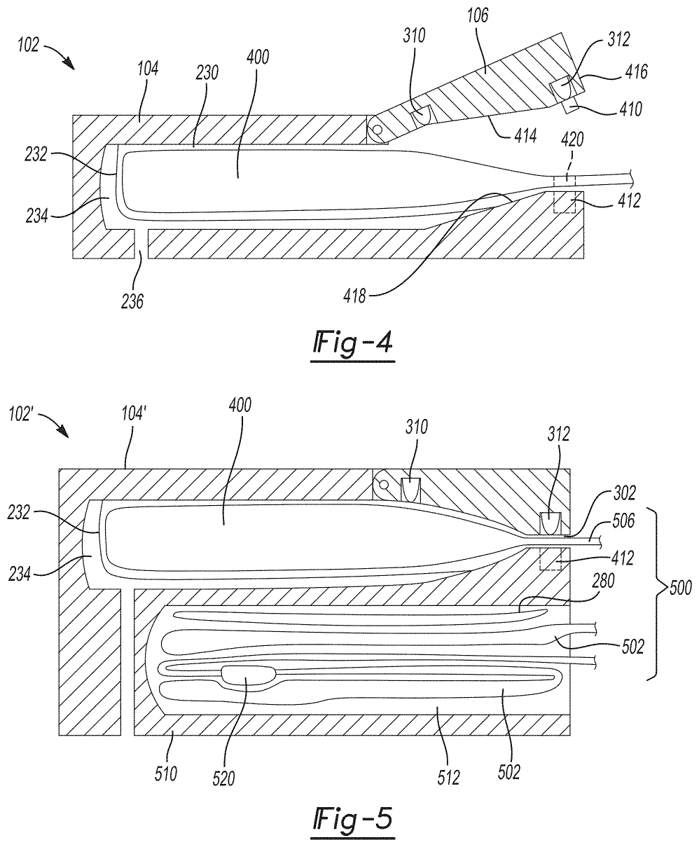

Each of the separation cells 102 may further include a lid 106. In at least one example embodiment, the lid 106 may be a hinged lateral lid. In at least one example embodiment, the lid 106 includes an upper portion of an external wall of the container 104. The lid 106 is dimensioned to allow, when open, an easy loading of a separation bag (see, e.g., separation bag 400 of FIGS. 4-5) containing (or full of) liquid into the separation cell 102. The container 104 may include a fast locking means (not shown) by which the lid 106 can be secured or locked to a remaining part of the container 104.

In at least one example embodiment, each of the separation cells 102 further includes first and second pinch valves 108, 110 (collectively referred to as the “pair of pinch valves 108, 110”).

The rotor 100 may further include a storage means, such as a central container 120. In at least one example embodiment, central container 120 may be subdivided into a plurality of satellite containers 122, as shown. The satellite containers 122 may be arranged about a central cavity 124.

The rotor 100 may further include a turntable 130. In at least one example embodiment, the turntable 130 may have a frustoconical shape. The rotor 100 may further include a manifold 132. The manifold 132 may have a generally circular shape or arrangement. In at least one example embodiment, the manifold 132 is arranged near a periphery 134 of the turntable 130. In at least one example embodiment the manifold 132 forms a ring within the turntable 130e (not shown). In at least one example embodiment, the manifold 132 may be referred to as a “peripheral circular manifold.”

Referring to FIG. 2, in at least one example embodiment, the rotor 100 may be part of a separation apparatus, such as a centrifuge 200. The rotor 100 may be supported by a bearing assembly 202 to facilitate rotation of the rotor 100 around a first or rotation axis 204. The rotor 100 may include a cylindrical rotor shaft 206 extending along a second or shaft longitudinal axis 208. A pulley 210 may be connected to the rotor shaft 206. The central container 120 may be connected to an upper end 212 of the rotor shaft 206. Accordingly, the shaft longitudinal axis 208 coincides with both the rotation axis 204 and a third or container longitudinal axis 214 of the central container 120.

In at least one example embodiment, the turntable 130 flares underneath an opening of the central container 120. The separation cells 102 may be mounted on the turntable 130 to form a symmetrical arrangement with respect to the rotation axis 204.

In at least one example embodiment, the centrifuge 200 further comprises a motor 220. The motor 220 may be coupled to the rotor 100 by a belt 222. The belt 222 may be engaged in a groove 224 of the pulley 210 to facilitate rotation of the rotor 100 about the rotation axis 204 by the motor 220.

Each of the separation cells 102 may define a median longitudinal axis 226. In at least one example embodiment, the separation cells 102 are mounted on the turntable 130 such that their respective median longitudinal axes 226 intersect the rotation axis 204, so that they are each located substantially at the same distance from the rotation axis 204, and/or the angles between their median longitudinal axes 226 are substantially the same (e.g., about 90 degrees). A position of the separation cells 102 on the turntable 130 may be adjusted so that the weight on the turntable is equally distributed when the separation cells 102 are empty, for example, to balance the rotor 100. In at least one example embodiment, the separation cells 102 are inclined with respect to the rotation axis 204 by an acute angle equal to the angle of a frustum of a cone that geometrically defines the turntable 130.

Each of the containers 104 defines a respective cavity 230. In at least one example embodiment, the cavity 230 is shaped and dimensioned to loosely accommodate a separation bag (see, e.g., separation bag 400 of FIGS. 4-5) containing (or full of) liquid. The cavity 230 (also referred to herein as the “separation compartment 230”) is defined by a bottom wall, which is the farthest from the rotation axis 204, a lower wall that is the closest to the turntable 130, an upper wall opposite to the lower wall, and two lateral walls.

In at least one example embodiment, the cavity 230 includes a main part, extending from the bottom wall, which has substantially the shape of a rectangular parallelepiped with rounded angles, and an upper part, which has substantially the shape of a prism having convergent triangular bases. Accordingly, the upper part of the cavity 230 may be defined by two pairs of opposite walls converging towards the central median axis 226 of the cavity 230. In at least one example embodiment, this design may facilitate a radial dilatation of the thin layer of a minor component of a composite fluid (e.g. the platelets in whole blood) after separation by centrifugation to make the minor component more easily detectable in the upper part of a separation bag (see, e.g., separation bag 400 of FIGS. 4-5).

The centrifuge 200 further includes a component transferring means for transferring at least one separated component from each separation bag (see, e.g., separation bag 400 of FIGS. 4-5) into a satellite bag (see, e.g., satellite bags 502 of FIG. 5) connected thereto. The component transferring means may, in at least one example embodiment, include a squeezing system for squeezing the separation bags within the separation compartments 230 and causing the transfer of separated components into the satellite bags, as discussed in greater detail below.

In at least one example embodiment, the squeezing system includes a flexible diaphragm 232 (also referred to herein interchangeably as a “bladder”) that is selectively coupled to a respective one of the containers 104 to define an expandable chamber 234 in a cavity thereof. More specifically, the flexible diaphragm 232 may be dimensioned to line the bottom wall of the cavity 230 and at least a portion (e.g., a large portion) of the lower wall of the cavity 230, which is the closest to the turntable 130. Each of the containers 104 may include a respective flexible diaphragm 232.

In at least one example embodiment, the squeezing system further includes the manifold 132. Each of the expandable chambers 234 is fluidly connected to the manifold 132 by a supply channel 236 that extends through a wall of the respective container 104, near the bottom thereof.

In at least one example embodiment, the squeezing system further includes a hydraulic pumping station 240. The hydraulic pumping station 240 may be configured to pump a hydraulic liquid in and/or out each of the expandable chambers 234 within the separation cells 102. The hydraulic liquid is selected to have a density slightly higher than the density of the densest of the components in the composite liquid to be separated (e.g., the red blood cells, when the composite liquid is blood). As a result, during centrifugation, the hydraulic liquid within each of the expandable chambers 234, whatever the volume thereof, will generally remain in the most external part of each of the separation cells 102. In at least one example embodiment, the hydraulic pumping station 240 is connected to each of the expandable chambers 234, through a rotary seal or fluid coupling 242, by a duct 244 that extends through the rotor shaft 206, the bottom and lateral wall of the central container 120, and, from a rim of the central container 120, radially through the turntable 130 where it connects to the manifold 132.

In at least one example embodiment, the hydraulic pumping station 240 includes a piston pump having a piston 246 movable in a hydraulic cylinder 248 fluidly connected via the rotary seal 242 to the duct 244. The piston 246 may be actuated by a stepper motor 250 that moves a lead screw 252 linked to a rod of the piston 246. The hydraulic cylinder 248 is also connected to a hydraulic liquid reservoir 254. The hydraulic liquid reservoir 254 may have access controlled by a valve 256. The valve 256 may be configured to selectively allow the introduction or the withdrawal of hydraulic liquid into and from a hydraulic circuit including the hydraulic cylinder 248, the duct 244, and the expandable chambers 234. In at least one example embodiment, a pressure gauge 258 is connected to the hydraulic circuit for measuring the hydraulic pressure therein.

In at least one example embodiment, the centrifuge 200 further includes four pairs of the first and second pinch valve members 108, 110. The pairs of pinch valve members 108, 110 may be mounted on the rotor 100 around the opening of the central container 120. Each of the pairs of pinch valve members 108, 110 may face a respective one of the separation cells 102 with which it is associated. The pinch valve members 108, 110 may be configured to facilitate selective blocking or allowing of a flow of liquid through a flexible plastic tube (see, e.g., tube 506 of FIG. 5), and selectively sealing and cutting the flexible plastic tube.

Each of the pinch valve members 108, 110 includes an elongated cylindrical body and a head having a groove 260. The groove 260 may be defined by a stationary upper jaw (not shown) and a lower jaw (not shown) movable between an open and a closed position. The groove 260 is dimensioned such that the tube (see, e.g., tube 506 of FIG. 5) of one or more bag sets can be snuggly engaged therein when the lower jaw is in the open position. The elongated body may include a mechanism (not shown) for moving the lower jaw and it is connected to a radio frequency generator that supplies the energy necessary for sealing and cutting the tube, which may be plastic.

In at least one example embodiment, the pinch valve members 108, 110 are mounted inside the central container 120, adjacent the interior surface thereof, so that their longitudinal axes are substantially parallel to the rotation axis 204 and their heads protrude above the rim of the central container 120. In at least one example embodiment, electric power is supplied to the pinch valve members 108, 110 through a slip ring array 270 that is mounted around a lower portion of the rotor shaft 206.

In at least one example embodiment, the centrifuge 200 further includes a first balancing means for initially balancing the rotor 100 when the weights of the separation bags (see, e.g., separation bag 400 of FIGS. 4-5) contained in the separation cells 102 are different. The first balancing means includes substantially the same structural elements as the elements of the component transferring means described above, namely: four expandable hydraulic chambers (e.g., similar to hydraulic chamber 234) interconnected by a manifold (e.g., similar to the manifold 132), and a hydraulic pumping station (e.g., similar to the hydraulic pumping station 240) for pumping hydraulic liquid into the hydraulic chambers through a duct (e.g., similar to the duct 244) connected to the manifold. In order to initially balance the rotor 100, whose four separation cells 102 contain four discrete volumes of a composite liquid that may not have the same weight (because the four volumes may be not equal, and/or the density of the liquid may slightly differ from one volume to the other one), the hydraulic pumping station is controlled so as to pump into the interconnected hydraulic chambers, at the onset of a separation process, a predetermined volume of hydraulic liquid that is so selected as to balance the rotor 100 in the most unbalanced situation. For whole blood, the determination of this balancing volume takes into account the maximum difference in volume between two blood donations, and the maximum difference in hematocrit (i.e., in density) between two blood donations. Under centrifugation forces, the hydraulic liquid will distribute unevenly in the four separation cells 102 depending on the difference in weight of the separation bags and balance the rotor 100. To achieve a desired or optimal initial balancing, the volume of the cavity 230 of the separation cells 102 may be selected so that the cavities 230, whatever the volume of the separation bags contained therein, are not full after the determined amount of hydraulic liquid has been pumped into the interconnected expandable chambers 234.

In at least one example embodiment the centrifuge 200 further includes a second balancing means for balancing the rotor 100 when the weights of the components transferred into satellite bags (see, e.g., satellite bags 502 of FIG. 5) in the central container 120 are different. For example, when two blood donations have the same hematocrit and different volumes, the volumes of plasma extracted from each donation are different, and the same is true when two blood donations have the same volume and different hematocrit. In at least one example embodiment, the second balancing means includes four flexible rectangular pouches 280 that are interconnected by four tube sections (not shown) each tube section connecting two adjacent pouches 280 by the bottom thereof. The pouches 280 contain a volume of balancing liquid having a density close to the density of the composite liquid. The volume of balancing liquid is so selected as to balance the rotor 100 in the most unbalanced situation. The four pouches 280 are dimensioned as to line the inner surface of the central container 120 and to have an internal volume that is larger than the volume of balancing liquid so that the balancing liquid can freely expand in any of the pouches 280. In operation, if, for example, four satellite bags (see, e.g., satellite bags 502 of FIG. 5) respectively adjacent to the four pouches 280 receive different volumes of a plasma component, the four satellite bags will press unevenly, under centrifugation forces, against the four pouches 280 which will result in the balancing liquid becoming unevenly distributed in the four pouches 280 and compensating for the difference in weight in the satellite bags.

In at least one example embodiment, the centrifuge further includes a controller 290 including a control unit (e.g., a microprocessor, a controller, etc.) and a memory (e.g., computer readable memory, etc.) for providing the microprocessor with information and programmed instructions relative to various separation protocols (e.g., a protocol for the separation of a plasma component and a blood cell component, or a protocol for the separation of a plasma component, a platelet component, and a red blood cell component) and to the operation of the apparatus in accordance with such separation protocols. In particular, the microprocessor is programmed for receiving information relative to the centrifugation speed(s) at which the rotor 100 is to be rotated during the various stages of a separation process (e.g., stage of component separation, stage of a plasma component expression, stage of suspension of platelets in a plasma fraction, stage of a platelet component expression, etc.), and information relative to the various transfer flow rates at which separated components are to be transferred from the separation bag (see, e.g., separation bag 400 of FIGS. 4-5) into the satellite bags (see, e.g., satellite bags 502 of FIG. 5). The information relative to the various transfer flow rates can be expressed, for example, as hydraulic liquid flow rates in the hydraulic circuit, or as rotation speeds of the stepper motor 250 of the hydraulic pumping station 240. The microprocessor may be further programmed for receiving, directly or through the memory, information from the pressure gauge 258 and from the four pairs of sensors 310, 312 and for controlling the centrifuge motor 220, the stepper motor 250 of the hydraulic pumping station 240, and the four pairs of pinch valve members 108, 110 to cause the separation apparatus to operate along a selected separation protocol.

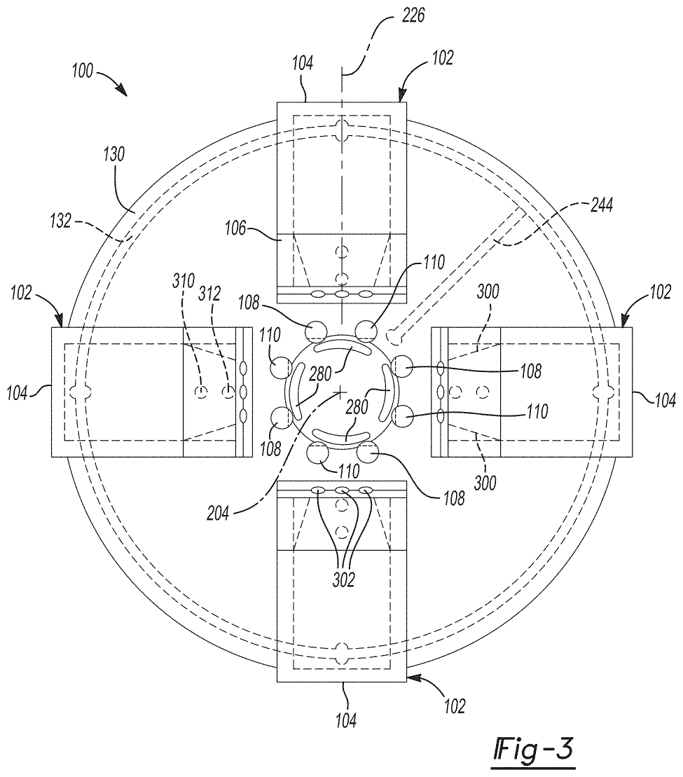

Referring to FIG. 3, in at least one example embodiment, an upper portion of each of the separation cells 102 includes walls 300 that converge toward the respective median longitudinal axis 226. In at least one example embodiment, the walls 300 converge toward a plurality (e.g., three, as shown) of channels 302 opening at the top of the container 104. The channels 302 may be cylindrical channels. The channels 302 may extend substantially parallel to one another.

In at least one example embodiment, the centrifuge 200 further includes four pairs of sensors for monitoring the separation of the various components occurring within each separation bag when the apparatus operates. Each pair of sensors includes a first or bag sensor 310 and a second or tube sensor 312. The sensors 310, 312 may be embedded in the lid 106 of the container 104 of each respective separation cell 102. The sensors 310, 312 may be mounted along the median longitudinal axis 226 of the container 104. The bag sensor 310 may be located the farther from the rotation axis 204 than the tube sensor 312. In at least one example embodiment, when a separation bag (see, e.g., separation bag 400 of FIGS. 4-5) rests in the container 104 and the lid 106 is closed, the bag sensor 310 faces an upper triangular part of the separation bag and the tube sensor 312 faces a proximal end of the tube (see, e.g., tube 506 of FIG. 5). The bag sensor 310 may be configured to detect blood cells in a liquid. The tube sensor 312 may be configured to detect the presence of absence of liquid in the tube as well as to detect blood cells in a liquid. Each of the sensors 310, 312 may include a photocell including an infrared LED and a photo-detector. In at least one example embodiment, electric power is supplied to the sensors 310, 312 through the slip ring array 270 (shown in FIG. 2) that is mounted around the lower portion of the rotor shaft 206 (shown in FIG. 2).

With reference to FIG. 4, each of the containers 104 may be configured to contain a respective separation bag 400. The container 104 may include a securing means for securing the separation bag 400 within the separation cell 102. In at least one example embodiment, the securing means includes one or more (e.g., two, as shown) pins 410 and one or more respective recesses 412. The pins 410 may protrude from an internal surface 414 of the lid 106, near a top 416 of the separation cell 102. The recesses 412 may be defined in an internal surface 418 of the container 104. The pins 410 may be spaced apart and dimensioned to be received in one or more respective holes 420 in an upper edge (e.g., the two upper corners) of the separation bag 400.

In at least one example embodiment, the centrifuge 200 (shown in FIG. 2) is configured to be used with a set of bags 500. With reference to FIG. 5, the set of bags 500 includes the separation bag 400 and a plurality of satellite bags 502. The separation and satellite bags 400, 502 may be flexible. In at least one example embodiment, the separation bag 400 is used successively for collection separately or away from the centrifuge 200 and then for separation within the centrifuge 200. The satellite bags 502 may be used within the centrifuge 200 for receipt of separated components.

In at least one example embodiment, the separation bag 400 is connecting to a tube (not shown), which may optionally have a needle (not shown) at its distal end for blood donation. The satellite bags 502 may be connected to the separation bag 400 by respective tubes 506 and optional breakable stopper(s) (not shown) between the separation bag 400 and respective satellite bags 502. The pinch valve members 108, 110 (shown in FIGS. 1-3) may be located on the respective tubes 506 leading to the satellite bags 502.

In at least one example embodiment, as shown in FIG. 5, a separation cell 102′ includes a container 104′ for the separation bag 400 is integral with a satellite bag container 510. The separation cell 102′ and the container 104′ are the same as the separation cell 102 and container 104 of FIGS. 1-3 except as otherwise described below. The satellite bag container 510 may define a satellite cavity 512 having the shape of a rectangular parallelepiped. The satellite cavity 512 may be configured to contain one of the pouches 280 of the balancing assembly described above. The separation bag container 104′ may be superimposed on the satellite bag container 510 so that the openings of both containers 104′, 510 are in the same plane, facing the rotation axis 204 (shown in FIGS. 2-3) when the container arrangement is mounted on the turntable 130 (shown in FIGS. 1-3).

In at least one example embodiment, the set of bags 500 includes the separation bag 400 for receiving a discrete volume of whole blood from a donor and two satellite bags 502, a first of the satellite bags configured to receive a plasma component and a platelet component from the whole blood and a second of the satellite bags to receive a red blood cell component of the whole blood. In at least one other example embodiment, the plurality of bags 500 includes the separation bag 400 for receiving a discrete volume of whole blood from a donor and three satellite bags 502, a first of the satellite bags configured to receive a plasma component of the whole blood, a second of the satellite bags configured to receive a platelet component of the whole blood, and a third of the satellite bags to receive a red blood cell component of the whole blood. In at least one other example embodiment, the plurality of satellite bags 502 includes more than three satellite bags. The set of bags 500 may include three-way connectors on tubing between the separation bag 400 and a portion of the satellite bags 502. When the separation bag 400 is in the container 104′, the tubes 506 may extend through respective channels 302. One or more of the satellite bags 502 may be fluidly connected to a filter 520, such as a leuko-reduction filter.

While described in conjunction with at least one arrangement of separation cells 102 of the centrifuge 200, it should be appreciated that the embodiments of the selective coupling assembly described herein may be utilized in any fluid processing, separating, and/or analysis system and is not limited to the separation apparatus described herein. Additionally or alternatively, embodiments of the present disclosure may be employed in any coupling between components in a bucket, or any partially confined volume, where access is limited. For instance, the bucket may include a deep well, or cavity that is only accessible on one side, is small in cross-sectional area, narrow in width or length, or has limited volume. In some examples, the bucket may be sized that a technician cannot place their hands into the cavity and reach the bottom wall of the bucket. In any case, the selective coupling assembly described herein can be used to quickly couple one component to another inside a bucket having limited volume and space.

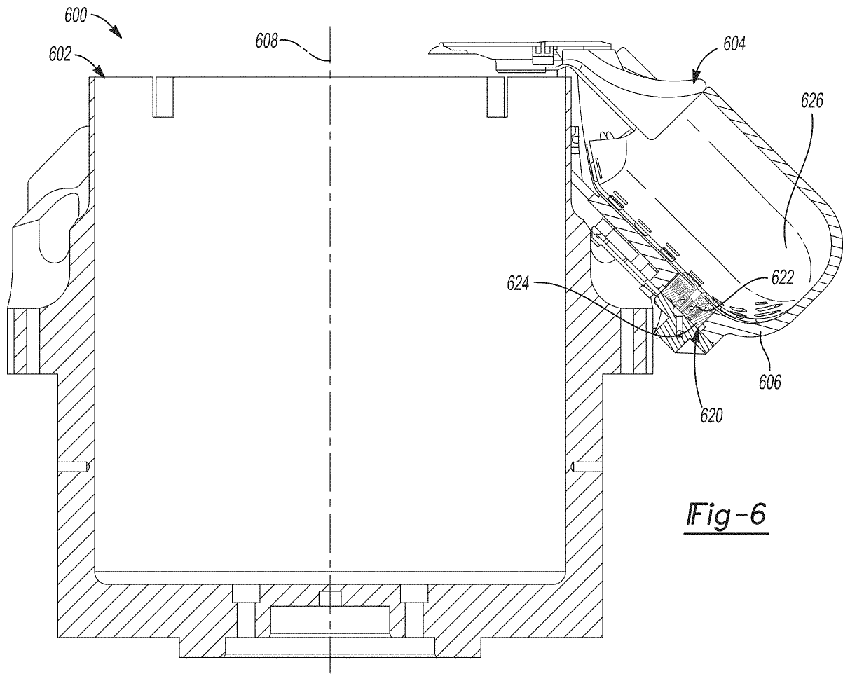

Referring to FIG. 6, a separation apparatus 600 according to at least one example embodiment is provided. The separation apparatus 600 is the same as the separation apparatus 200 of FIG. 2 except for presence of a selective coupling assembly that couples a bladder to a bucket, as described below.

In at least one example embodiment, the separation apparatus 600 includes a rotor 602 and at least one separation cell 604 (e.g., a single separation cell 604, as shown). The separation cell 604 includes a bucket 606 (also referred to as a container or bladder holder). As described above, the bucket 606 of the separation cell 604 may be disposed at an angle relative to a rotation axis 608 of the rotor 602. The separation apparatus 600 includes a selective coupling assembly 620 (also referred to as a “quick connector,” a “QC connector,” or an “interconnection assembly”). The selective coupling assembly 620 includes a connector 622 and a receiver 624. In at least one example embodiment, the connector 622 is associated with a flexible diaphragm or bladder 626 and the receiver 624 is associated with the bucket 604.

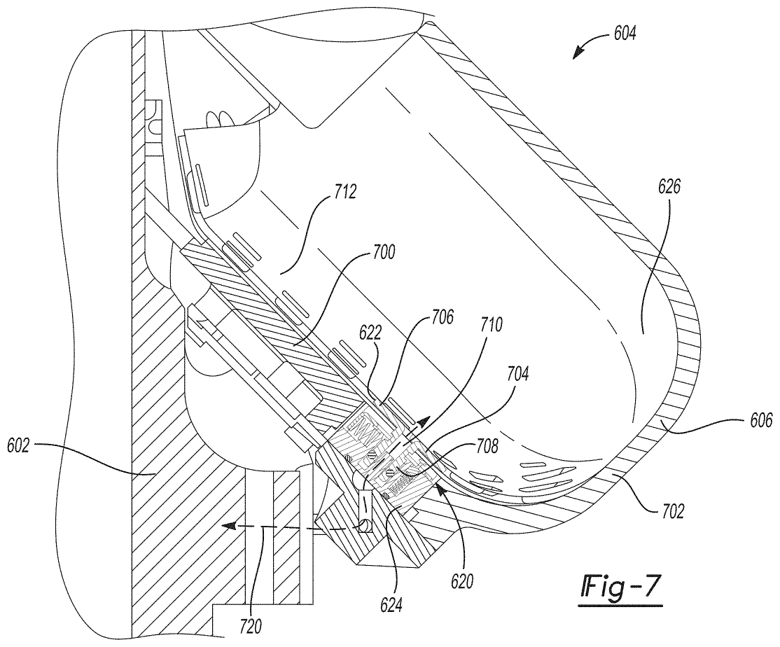

As shown in FIG. 7, the receiver 624 of the selective coupling assembly 620 may be attached to at least one wall 700 of the bucket 606. In at least one example embodiment, the receiver 624 may be at least partially in the wall 700 of the bucket. The receiver 624 may be disposed adjacent to a bottom wall 702 of the bucket 606. The receiver 624 may be made from a polymer (e.g., plastic), metal, or any combination thereof. In at least one example embodiment, a metal of the receiver 624 may include aluminum, steel, titanium, or any combination thereof. The receiver 624 may include locking elements, a lumen passing therethrough, and/or one or more external seals, as will be described in greater detail below.

The connector 622 is shown attached to the bladder 626. In at least one example embodiment, the connector 622 includes a body 704 having a flat base 706 and an engaging plug 708 extending from the flat base 706. The connector 622 may have or define a first lumen 710 passing therethrough.

The connector 622 may be formed from the bladder 626, or otherwise attached to the bladder 626. For instance, the flat base 706 of the connector 622 may be welded (e.g., ultrasonically) and/or adhered with an adhesive to the bladder 626. In at least one example embodiment, the connected is formed from a polymer (e.g., a plastic material).

In at least one example embodiment, the flat base 706 of the connector 622 may be captured between inner and outer walls, or layers, of the bladder 626 and affixed therebetween. For instance, a wall 712 of the bladder 626 may correspond to a laminate having one or more layers. In this case, a hole may be formed through the layers and the flat base 706 may be between two or more layers in the laminate. The layers of the laminate may then be attached to the flat base 706 via, gluing with an adhesive, melting, and/or welding using RF or ultrasonics. When attached, the interior volume of the bladder 626 is sealed from an exterior of the bladder 626 around and in contact with the flat base 706. However, the first lumen 710 may pass through the engaging plug 708, providing a fluid path 720 into the interior volume of the bladder 626 within a defined space.

Once connected to the receiver 624, the fluid flow path 720 is formed between a fluid source (e.g., a liquid or gas supply) and an interior volume of the bladder 626, or vice versa. The fluid flow path 720 may be unimpeded at the interconnection of the selective coupling assembly 620. In at least one example embodiment, there may be no valves or seals disposed along the fluid flow path within the selective coupling assembly 620. That is, the fluid flow path 720 may be free of valves and seals.

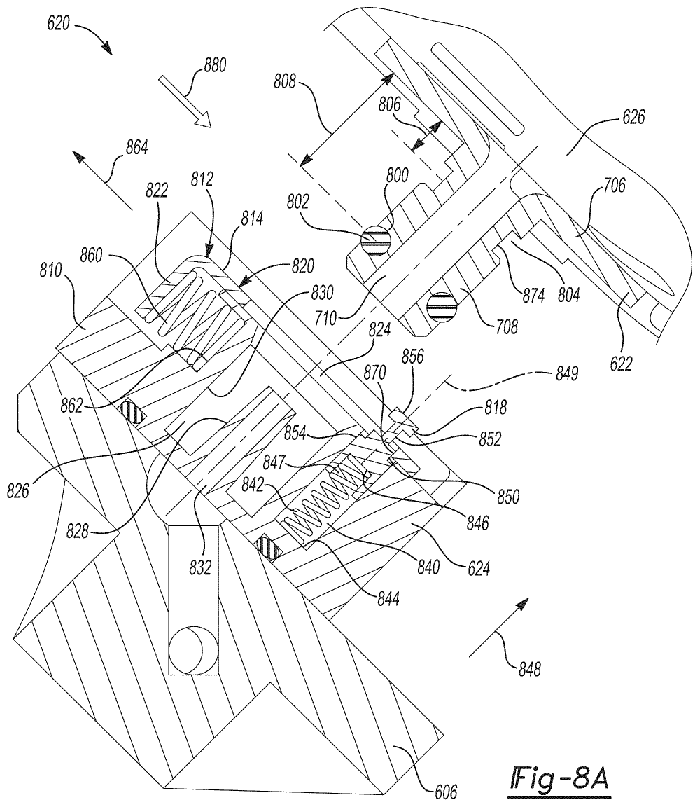

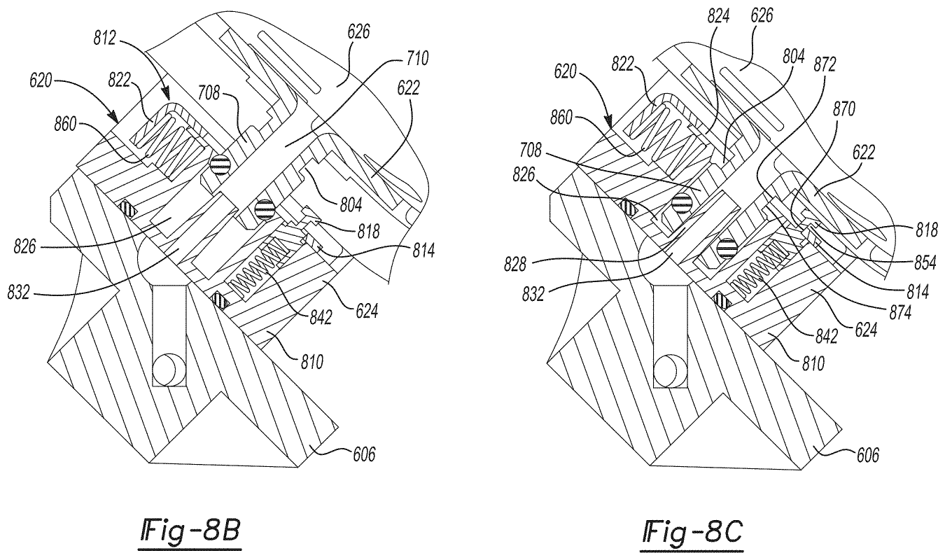

FIGS. 8A-8C show the selective coupling assembly 620 in various states of engagement and disengagement according to at least one example embodiment. The connector 622 includes the engaging plug 708 that protrudes from the flat base 706. The engaging plug 708 may define a first annular groove 800 (also referred to herein as the “O-ring groove 800”) that at least partially receives and contains (or is configured to at least partially receive and contain) an O-ring 802. When the engaging plug 708 is engaged with the receiver 624, the O-ring 802 of the connector 622 may reduce or prevent leaking of hydraulic fluid or pneumatic gas flowing along the fluid flow path 720 (shown in FIG. 7) between the fluid source and the interior volume of the bladder 626. The engaging plug 708 may further define a locking recess 804, which may be an annular groove, as shown. An axial center of the locking recess 804 may be a first distance 806 from the flat base 706. An axial center of the annular groove 800 may be a second distance 808 from the flat base 706. The second distance 808 may be greater than the first distance 806.

In at least one example embodiment, the receiver 624 generally includes a base 810 and an automatic latch assembly, such as a spring-loaded latch 812. The spring-loaded latch 812 may include a latch plate 814 and a trigger pin 818. The latch plate 814 may be slidably attached to the base 810. The latch plate 814 includes a main portion 820 and a flange 822. The main portion 820 defines a receiving aperture 824. The main portion 820 may be configured to receive at least a portion of the engaging plug 708 of the connector 622. The main portion 820 is further configured to receive at least a portion of the trigger pin 818.

The base 810 has or defines a first or connector receptacle 826. In at least one example embodiment, the base 810 includes an inner annular protrusion or barb 828. The connector receptacle 826 may be defined in a region radially between an annular wall 830 and the inner annular protrusion 828. The inner annular protrusion 828 may define a second or receiver lumen 832. The receiving aperture 824 of the latch plate 814 may be concentrically around the connector receptacle 826 and the second lumen 832.

The base 810 further defines a second or trigger pin receptacle 840. The trigger pin receptacle 840 receives at least a portion of the trigger pin 818. A first spring 842 (e.g., a compression spring) is at least partially received in the trigger pin receptacle 840. The first spring 842 engages the base 810 and the trigger pin 818. In at least one example embodiment, the first spring 842 extends between a bottom wall 844 of the trigger pin receptacle 840 and a bottom surface 846 of the trigger pin 818. In at least one example embodiment, the trigger pin 818 has or defines a recess 847. The recess 847 is at least partially defined by the bottom surface 846 of the trigger pin 818. The recess 847 may be configured to receive a portion of the first spring 842. In at least one example embodiment, the first spring 842 is configured to bias the trigger pin 818 in a first or outward direction 848. The first direction 848 may be substantially parallel to a longitudinal axis 849 of the trigger pin 818.

In at least one example embodiment, the trigger pin 818 has or defines a first stepped portion or annular groove 850 and a second portion or annular groove 852. The first annular groove 850 may be closer to the bottom surface 846 than the second annular groove 852. That is, a distance between the second annular groove 852 and the bottom surface 846 may be longer than a distance between the first annular groove 850 and the bottom surface 846. The trigger pin 818 may define a first diameter at the first annular groove 850, a second diameter at the second annular groove 852, and a third diameter at an outermost surface 854. The first diameter may be smaller than the third diameter. The second diameter may be smaller than the first diameter (and the third diameter). The trigger pin 818 includes a top surface 856.

The receiver 624 may further include a second spring 860 (e.g., a compression spring). The second spring 860 may engage the base 810 and the latch plate 814. The second spring 860 may be between the flange 822 of the latch plate 814 and an outer surface 862 of the base 810. The second spring 860 may be configured to bias the latch plate 814 in a second or lock direction 864. The second direction 864 may be substantially perpendicular to the first direction 848.

The spring-loaded latch 812 may be configured to move between a release, disengaged, or unlocked position (shown in FIG. 8A) and a lock or engaged position or state (shown in FIG. 8C). In the release position, the latch plate 814 may be held open such that the receiving aperture 824 is capable of receiving the engaging plug 708 of the connector 622. For instance, a first annular groove 850 of the trigger pin 818 may receive a first or pin edge 870 of the latch plate 814 in the release position. When the trigger pin 818 is moved along a line parallel to the longitudinal axis 849, the first annular groove 850 of the trigger pin 818 may be moved into the receiver 624 and second annular groove 852 the trigger pin 818 may be positioned in line with the latch plate 814. This positioning of the trigger pin 818 allows the second spring 860 to translate, slide, or otherwise move the latch plate 814 in the second direction 864 from the release position (FIG. 8A) to the latch position (FIG. 8C). After translation of the latch plate 814, the first edge 870 of the latch plate 814 is at least partially within the second annular groove 852.

In FIG. 8C, the engaging plug 708 of the connector 622 is shown locked to the latch plate 814 of the receiver 624. In at least the example embodiment shown, this locking may occur by a second or locking edge 872 of the latch plate 814 engaging with a plug surface 874 of the engaging plug 708 (as shown in FIG. 8C). The latch plate 814, including the second edge 872, may be at least partially in the locking recess 804 of the connector 622.

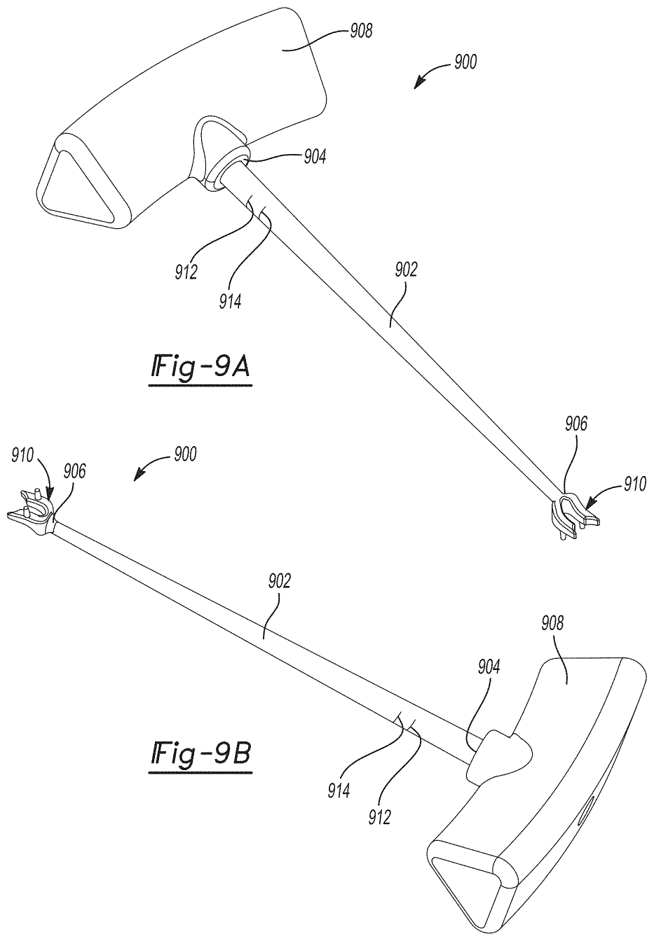

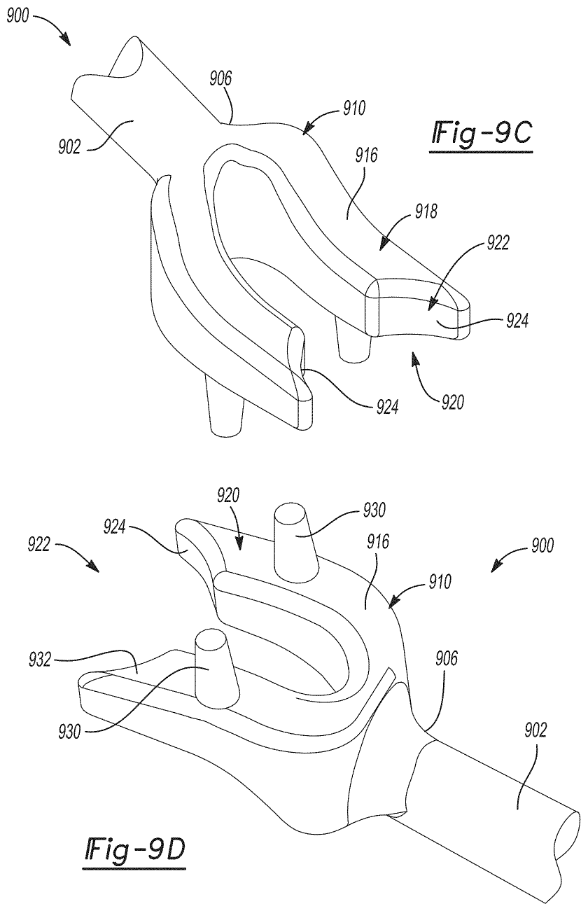

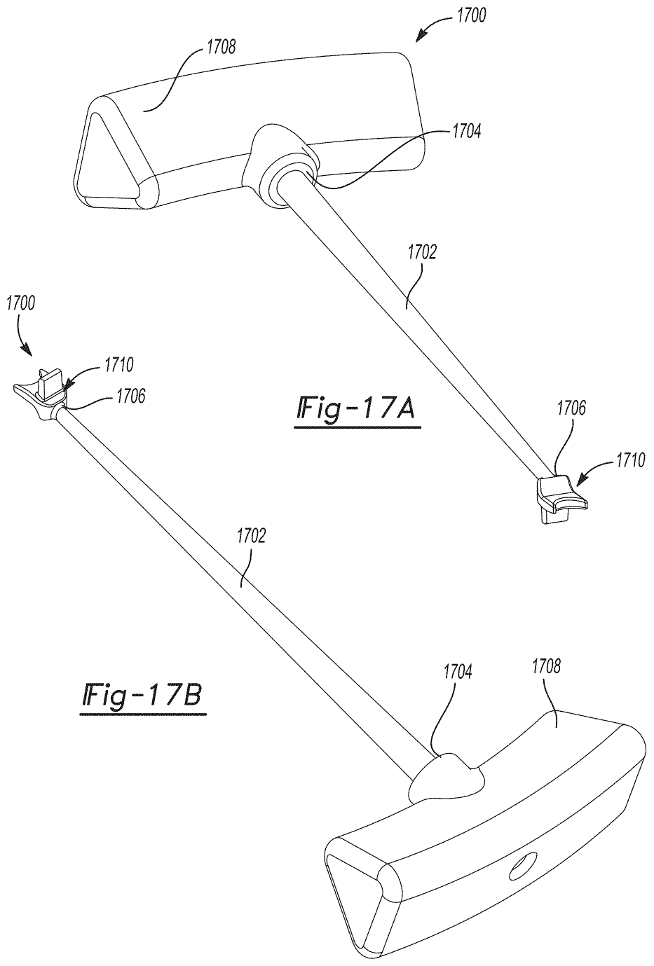

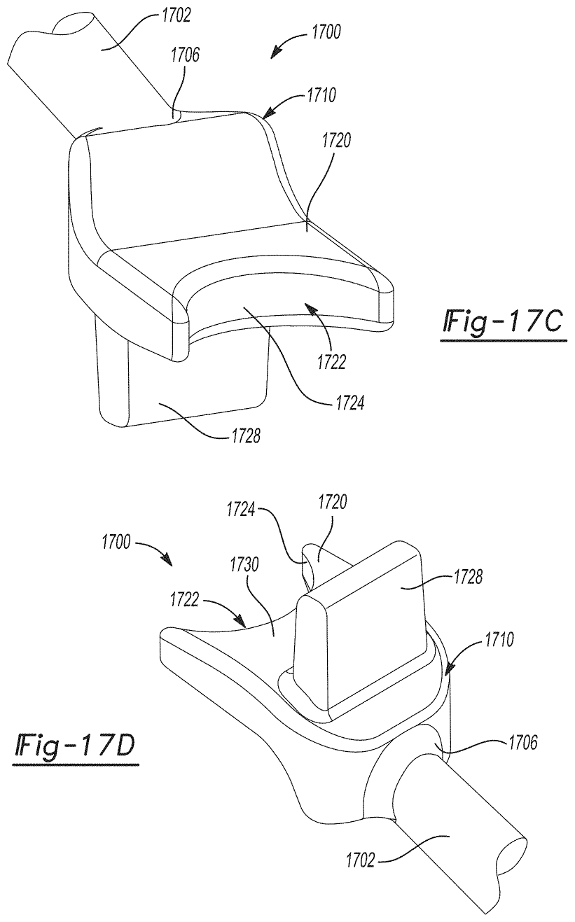

FIG. 8A shows a schematic cross-section detail view of the connector 622 and the receiver 624 of the selective coupling assembly 620 in the release state. During coupling of the connector 622 to the receiver 624, the engaging plug 708 may be supported or guided by a tool (see, e.g., tool 900 of FIGS. 9A-9D and tool 1700 of FIGS. 17A-17D) inserted in a space between the bladder 626 and the bucket 606, as indicated by arrow 880 in FIG. 8A. Additional details of this guided alignment are described below. Among other things, the tool may allow a technician to align an axis of the engaging plug 708 with an axis of the connector receptacle 826 and/or second lumen 832. In some examples, however, a tool may not be required when coupling the connector 622 to the receiver 624 and/or decoupling the connector 622 from the receiver 624. In any event, the bladder 626 may be coupled or decoupled while the bladder 626 and integrated connector 622 are disposed within a receiving volume of a bladder holder (e.g., a bucket, etc.) where the receiver 624 is also disposed.

FIG. 8B illustrates an intermediate or partially engaged configuration in which the engaging plug 708 is partially within the connector receptacle 826 and the spring-loaded latch 812 is in the release configuration. As shown in FIG. 8B, the engaging plug 708 of the connector 622 is brought into contact with the connector receptacle 826 in the receiver 624 such that the first lumen 710 of the connector 622 is concentric with the second lumen 832 of the receiver 624. As the bladder 626 and connector 622 are moved toward the receiver 624 (e.g., in a seated position), at least a portion of the bladder 626 and/or connector 622 may contact the trigger pin 818 causing the latch plate 814 to move into an engaged position with the locking recess 804 of the engaging plug 708 as shown in FIG. 8C. In this position the connector 622 is prevented from moving along the axis of the first and second lumens 710, 832 and the bladder 626 is locked to the receiver 624 and bucket 606 at this location.

Once engaged, the bladder 626 may be separated from the bucket 606 by releasing the latch plate 814 of the receiver 624. Releasing the latch plate 814 may include inserting a tool (e.g., a coupling tool) in the space between the bladder 626 the wall 700 (shown in FIG. 7) where the receiver 624 is disposed, as will be described in greater detail below. The coupling tool may then engage with actuation features (e.g., holes, slots, tabs, etc.) disposed in the latch plate 814 and the coupling tool may be moved to release the engagement of the latch plate 814 with the recess, or groove, of the engaging plug. Referring to FIG. 8C, this movement would correspond to a movement direction running from the top left-hand side of the figure to the bottom right-hand side of the figure. When the latch plate 814 is moved a certain distance in this movement direction, the trigger pin 818 would translate toward the bladder 626 and lock the latch plate 814 in the release position (shown in FIGS. 8A-8B). The movement direction may be in a push or a pull direction, for example, depending on the arrangement of the receiver 624 in the bucket 606 and/or other design choices.

In some examples, the bladder 626 may be separated from the bucket 606 by releasing the latch plate 814 of the receiver 624 without requiring a separate tool. Releasing the latch plate 814 may include actuating the latch plate 814 of the receiver 624 such that the latch plate 814 disengages from the locking recess 804 of the engaging plug 708.

FIGS. 9A-9D illustrate a tool 900 (also referred to as the “selective coupling tool”) that may be used to facilitate locking and/or unlocking the connector 622 and associated bladder 626 from the receiver 624 and associated bucket 606 (shown in FIG. 6). The tool 900 includes a shaft 902 extending from a proximal end 904 to a distal end 906. A handle 908 may be disposed at the proximal end 904 of the shaft 902. A coupling end 910 may be disposed at the distal end 906 of the shaft 902.

In at least one example embodiment, the tool 900 may include indicia configured to indicate a depth of insertion in the bucket 606 (shown in FIG. 6). In the example embodiment shown, the shaft 902 may define first and second indicia 912, 914, as shown. The indicia 912, 914 may be longitudinally spaced apart from one another. In at least one example embodiment, the first indicium 912 indicates a depth of insertion for locking of the selective coupling assembly 620 (shown in FIG. 6) and the second indicium 914 indicates a depth of insertion for unlocking of the selective coupling assembly 620. In at least one example embodiment, the indicia are etched into the shaft 902.

In at least one example embodiment, as best shown in FIG. 9C, the tool 900 may include features that facilitate locking of the selective coupling assembly 620 (shown in FIG. 6). The coupling end 910 of the tool 900 may comprise a forked extension 916 protruding from the distal end 906 of the shaft 902. The forked extension 916 may be U-shaped, or in the shape of a horseshoe. The forked extension 916 may extend between a first side 918 (shown in FIG. 9C) of the coupling end 910 and a second side 920 (shown in FIG. 9D) of the coupling end 910.

The forked extension 916 may define a cradle 922 that is configured to engage with an outer surface of the engaging plug 708 (shown in FIG. 7) of the connector 622 (shown in FIG. 7), as will be described in greater detail below. The cradle 922 may include one or more arcuate contact surfaces 924 that are sized to match a size of the outer diameter of the engaging plug 708 of the connector 622. In at least one other example embodiment, a cradle may comprise one or more arcuate contact surfaces that are sized to have a diameter that is larger than the outer diameter of the engaging plug 708 of the connector 622. The cradle 922 may be used to assist a technician in guiding the engaging plug 708 into the receiver 624 (shown in FIG. 7).

In at least one example embodiment, as best shown in FIG. 9D, the tool 900 may include one or more release features that engage with the latch plate 814 (shown in FIG. 8A) to facilitate unlocking of the selective coupling assembly 620 (shown in FIG. 6). In at least one example embodiment, the release features include one or more release pins 930 that protrude from a surface 932 on the second side 920 of the coupling end 910 of the tool 900. In at least one example embodiment, the pins 930 may extend substantially perpendicular to the bottom surface 932 of the coupling end 910. The pins 930 are sized and shaped to engage with corresponding features (e.g., holes, slots, etc.) in the latch plate, as will be described in greater detail below. In at least one example embodiment, the release pins 930 have a frustoconical shape.

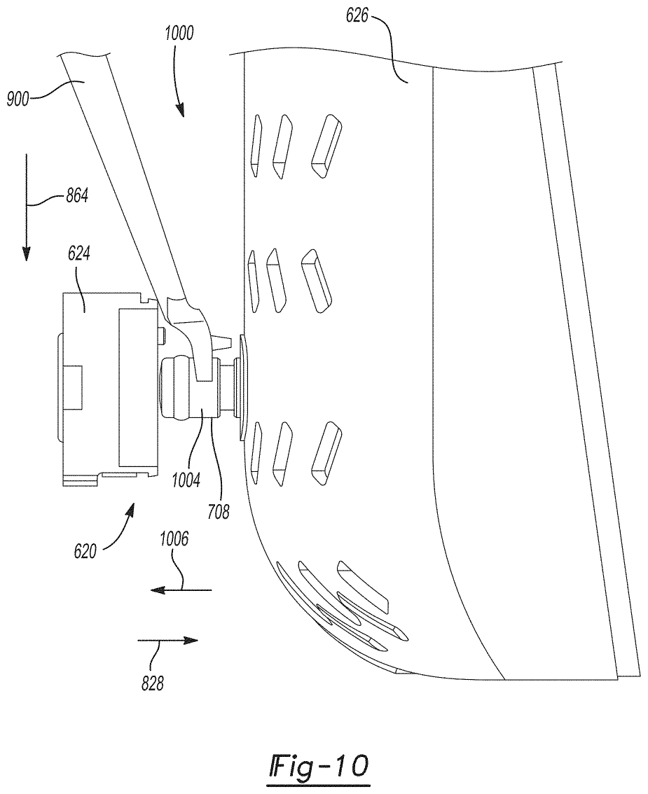

As discussed above, the tool 900 may be used to facilitate locking of the selective coupling assembly 620 (shown in FIG. 6). With reference to FIG. 10, an arrangement for locking the selective coupling assembly 620 according to at least one example embodiment is shown. After the bladder 626 is inserted into the bucket 606 (shown in FIG. 6), the tool 900 may be inserted into a region or cavity 1000 between the bladder 626 and the receiver 624. The tool 900 may be moved toward the selective coupling assembly 620. In at least the example embodiment shown, the tool 900 is moved in the second direction 864. The cradle 922 (shown in FIGS. 9C-9D) of the selective coupling tool 900 may receive an outer surface 1004 of the engaging plug 708 such that the arcuate contact surfaces 924 contact the outer surface 1004. The connector 622, together with the bladder 626, may be moved toward the receiver 624 in a third or inward direction 1006 opposite the first direction 848 (shown in FIG. 8A). During the movement in the third direction 1006, the tool 900 may guide the engaging plug 708 into the receiving aperture 824 of the receiver 624. When the engaging plug 708 is guided and inserted into the receiving aperture 824, the tool 900 may be removed from the region 1000 and the bladder 626 may be pushed further in the third direction 1006 into the lock or fully engaged state with the receiver 624.

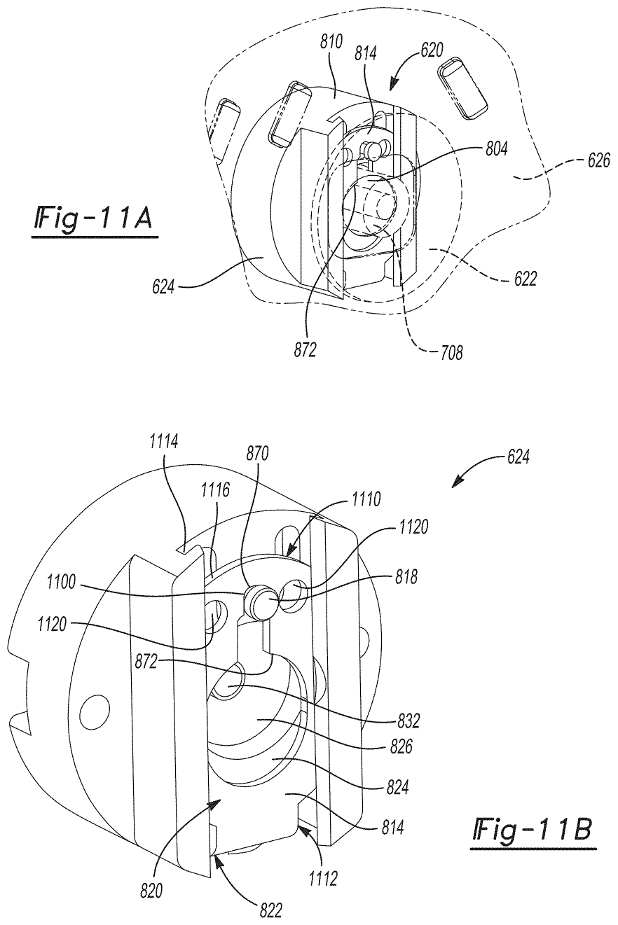

In the lock state, as shown in FIGS. 11A-11B and described above, the engaging plug 708 is at least partially in the receiving aperture 824 The pin edge 870 of the latch plate 814 is at least partially within the second annular groove 852 (shown in FIG. 8A) of the trigger pin 818. The locking edge 872 of the latch plate 814 is at least partially in the locking recess 804 of the engaging plug 708. This engagement axially locks the connector 622 to the receiver 624.

As best shown in FIG. 11B, the trigger pin 818 is at least partially in a slot 1100 of the latch plate 814. In the example embodiment shown, the slot 1100 is defined in the locking edge 872 such that the slot 1100 is in communication with the receiving aperture 824. The main portion 820 of the latch plate 814 extends between a first end 1110 and a second end 1112. The main portion 820 of the latch plate 814, including the first end 1110, is at least partially within a channel 1114 of the base 810. The first end 1110 may include an arcuate surface 1116. The flange 822 may extend from the second end 1112. The latch plate 814 defines one or more release apertures 1120.

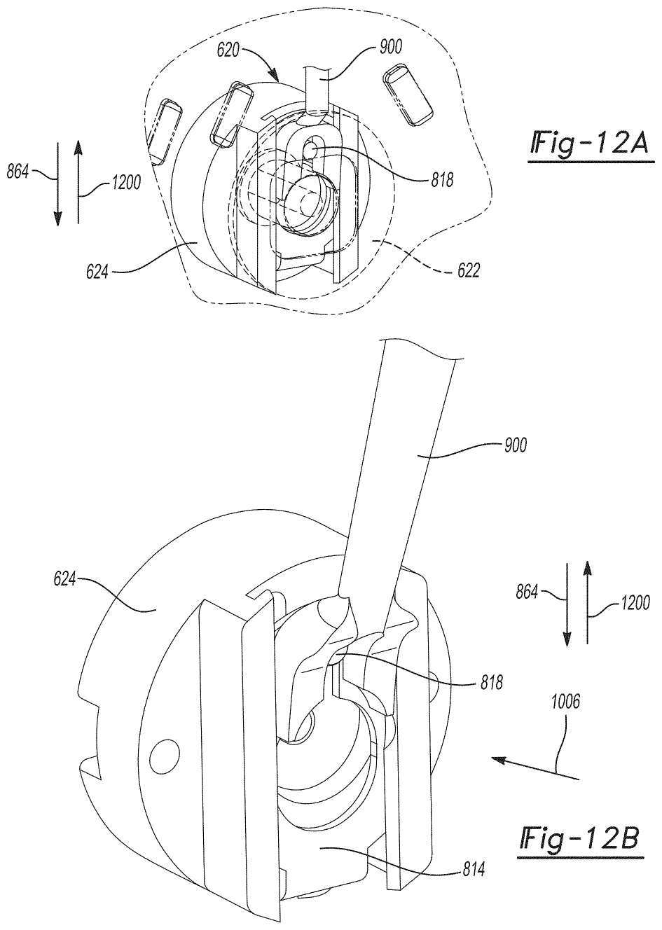

As discussed above, the tool 900 (shown in FIGS. 9A-9D) may be used to facilitate unlocking of the selective coupling assembly 620. With reference to FIGS. 12A-12B, the tool 900 may be moved toward the selective coupling assembly 620. In at least the example embodiment shown, the tool 900 is moved in the second direction 864 and inserted into the region 1000 (shown in FIG. 10) between the connector 622 and the receiver 624. The tool 900 may be further move in the third direction 1006 to insert the pins 930 (shown in FIGS. 9A and 9C) of the tool 900 at least partially into the release apertures 1120 (shown in FIG. 11B) of the latch plate 814. With the pins 930 in the release apertures 1120, the tool 900 may be translated in a fourth or unlock direction 1200 opposite the second direction 864. Engagement of the pins 930 of the tool 900 with the latch plate 814 causes the latch plate 814 to move in the fourth direction 1200 together with the tool 900.

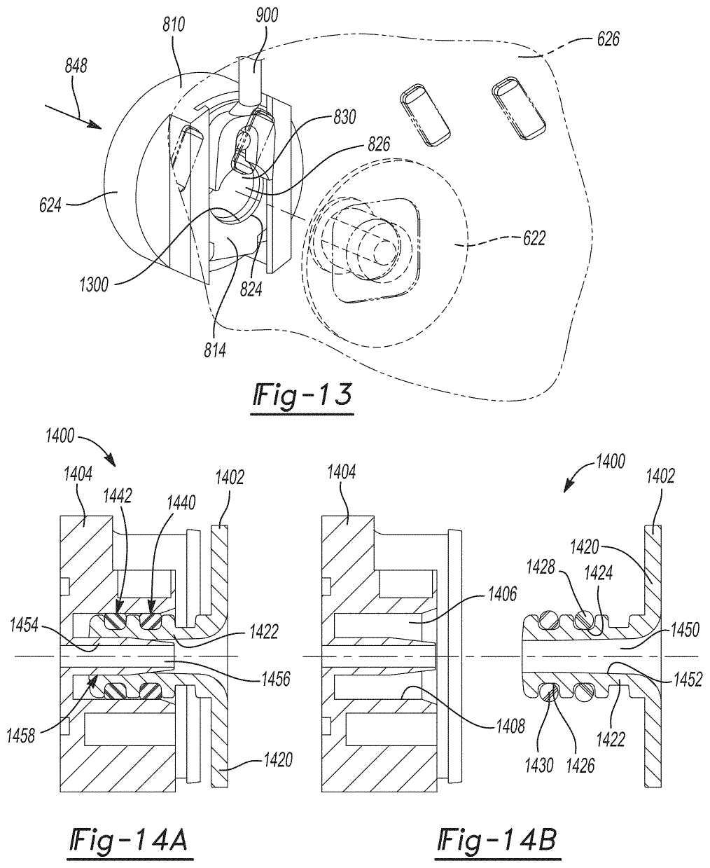

Referring to FIG. 13, the selective coupling assembly 620 is shown in the release state with the tool 900 still engaged with the latch plate 814. In the release state, the connector receptacle 826 of the base 810 is substantially concentric with the receiving aperture 824 of the latch plate 814. Stated another way, a plate edge 1300 may be moved closer to and even overlap, or coincide with, the annular wall 830 in the base 810 of the receiver 624. In this position, the engaging plug 708 of the connector 622 may be moved in the first direction 848 such that it is removed from the receiver 624, as shown in FIG. 13.

Although the connector 622 is shown having the O-ring 802 (shown in FIG. 8A) configured to form a seal against the annular wall 830 of the receiver 624, it should be appreciated that different or additional sealing elements or features may be used to reduce or prevent leaking of hydraulic fluid or pneumatic gas flowing along the fluid flow path 720 (shown in FIG. 7) between the fluid source and the interior volume of the bladder 626. For instance, the engaging plug may comprise a sealing feature (e.g., configured as an elastically bending, or flexible, ridge) and/or a compliant portion that is capable of providing a seal (e.g., air tight, liquid tight, etc.) between a connector and a receiver. In some examples, the sealing feature may be a separate component that is attached to an engaging plug, insert molded with the engaging plug, and/or co-molded with the engaging plug. In one example, the sealing feature may be integrally formed from the material of the engaging plug.

With reference to FIGS. 14A-14B, another selective coupling assembly 1400 according to at least one example embodiment is provided. The selective coupling assembly 1400 may be the same as the selective coupling assembly 620 of FIG. 6 except as otherwise provided below. The selective coupling assembly 1400 includes a connector 1402 and a receiver 1404. The receiver 1404 has or defines a connector receptacle 1406 (shown in FIG. 14B) configured to receive a portion of the connector 1402. The connector receptacle 1406 is at least partially defined by an annular wall 1408.

The connector 1402 includes a flat base 1420 and an engaging plug 1422. The engaging plug 1422 defines first and second annular grooves 1424, 1426 that at least partially receive first and second O-rings 1428, 1430. When the connector 1402 is engaged with the connector receptacle 1406, first and second annular seals 1440, 1442 are formed between the annular wall 1408 and the first and second O-rings 1428, 1430, respectively.

The connector 1402 defines a first or connector lumen 1448 that extends through both the flat base 1420 and the engaging plug 1422. An annular lumen surface 1450 at least partially defines the first lumen 1448. The receiver 1404 includes an inner annular protrusion or barb 1452. The inner annular protrusion 1452 protrudes into the connector receptacle 1406 and defines a second lumen 1454. When the connector 1402 is engaged with the receiver 1404, the inner annular protrusion 1452 is at least partially within the first lumen 1448. A third annular seal 1456 is formed between the inner annular protrusion 1452 and the lumen surface 1450. Accordingly, the selective coupling assembly 1400 includes both inner (i.e., the third seal 1456) and outer (i.e., the first and second seals 1440, 1442).

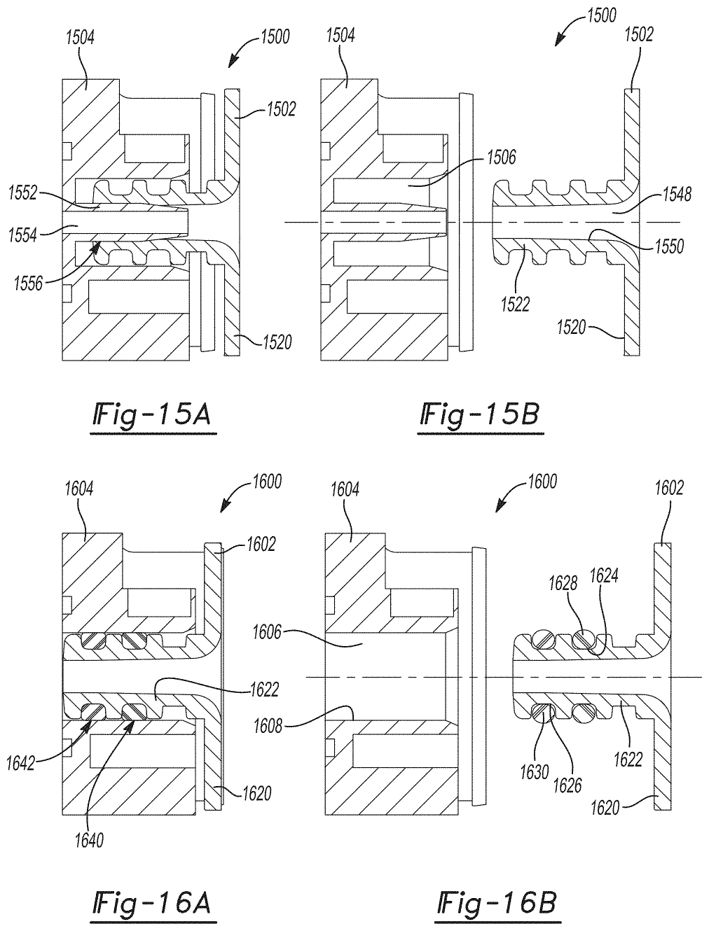

With reference to FIGS. 15A-15B, another selective coupling assembly 1500 according to at least one example embodiment is provided. The selective coupling assembly 1500 may be the same as the selective coupling assembly 620 of FIG. 6 except as otherwise provided below. The selective coupling assembly 1500 includes a connector 1502 and a receiver 1504. The receiver 1504 defines a connector receptacle 1506 (shown in FIG. 15B) configured to receive a portion of the connector 1502.

The connector 1502 includes a flat base 1520 and an engaging plug 1522. The connector 1502 defines a first lumen 1558 that extends through both the flat base 1520 and the engaging plug 1522. An annular lumen surface 1550 at least partially defines the first lumen 1548.

The receiver 1504 includes an inner annular protrusion or barb 1552. The inner annular protrusion 1552 protrudes into the connector receptacle 1506 and defines a second lumen 1554. When the connector 1502 is engaged with the receiver 1504, the inner annular protrusion 1552 is at least partially within the first lumen 1548. An annular seal 1556 is formed between the inner annular protrusion 1552 and the lumen surface 1550. Accordingly, the selective coupling assembly 1400 includes only an inner (i.e., the seal 1656).

With reference to FIGS. 16A-16B, another selective coupling assembly 1600 according to at least one example embodiment is provided. The selective coupling assembly 1600 may be the same as the selective coupling assembly 620 of FIG. 6 except as otherwise provided below. The selective coupling assembly 1600 includes a connector 1602 and a receiver 1604. The receiver 1604 defines a connector receptacle 1606 (shown in FIG. 16B) configured to receive a portion of the connector 1602. The connector receptacle 1606 is at least partially defined by an annular wall 1608.

The connector 1602 includes a flat base 1620 and an engaging plug 1622. The engaging plug 1622 defines first and second annular grooves 1624, 1626 that at least partially receive first and second O-rings 1628, 1630. When the connector 1602 is engaged with the connector receptacle 1606, first and second annular seals 1640, 1642 are formed between the annular wall 1608 and the first and second O-rings 1628, 1630, respectively. Accordingly, the selective coupling assembly 1600 includes only outer seals (i.e., the first and second seals 1640, 1642).

FIGS. 17A-17D illustrate a tool 1700 (also referred to as the “selective coupling tool”) that may be used to facilitate locking and/or unlocking a connector and associated bladder from a receiver and associated bucket. The tool 1700 includes a shaft 1702 extending from a proximal end 1704 to a distal end 1706. A handle 1708 may be disposed at the proximal end 1704. A coupling end 1710 may be disposed at the distal end 1706 of the shaft 1702. In at least one example embodiment, the tool 1700 mat further include indicia (see, e.g., indicia 1712, 1714 of FIGS. 9A-9B) configured to indicate a depth of insertion in a bucket.

In at least one example embodiment, as best shown in FIG. 17C, the tool 1700 may include features that facilitate locking of the selective coupling mechanism. The coupling end 1710 of the tool 1700 may comprise a forked extension 1720 protruding from the distal end 1706 of the shaft 702. The forked extension may be U-shaped, or in the shape of a horseshoe. The forked extension 1720 may define a cradle 1722 that is configured to engage with an outer surface of an engaging plug of the connector. The cradle 1722 may comprise one or more arcuate contact surfaces 1724 that are sized to match a size of the outer diameter of the engaging plug of the connector. In at least one other example embodiment, a cradle may comprise one or more arcuate contact surfaces that are sized to have a diameter that is larger than the outer diameter of the engaging plug of the connector. The cradle 1722 may be used to assist a technician in guiding the engaging plug into the.

In at least one example embodiment, as best shown in FIG. 17D, the tool 1700 may include one or more release features that engage with a latch plate to facilitate unlocking of the selective coupling mechanism. In at least one example embodiment, the release feature includes protrusion, such as a transverse plate 1730 that protrudes from a surface 1732 of the coupling end 1710 of the tool 1700. In at least one example embodiment, the plate 1730 may extend substantially perpendicular to the surface 1732 of the coupling end 1710. The plate 1730 is sized and shaped to engage with corresponding features (e.g., a flange) on the latch plate, as will be described in greater detail below.

As discussed above, the tool 1700 may be used to facilitate locking an/or unlocking of a selective coupling assembly. The tool 1700 may be used in the same manner as the tool 900 to facilitate locking a selective coupling assembly using the cradle 1722.

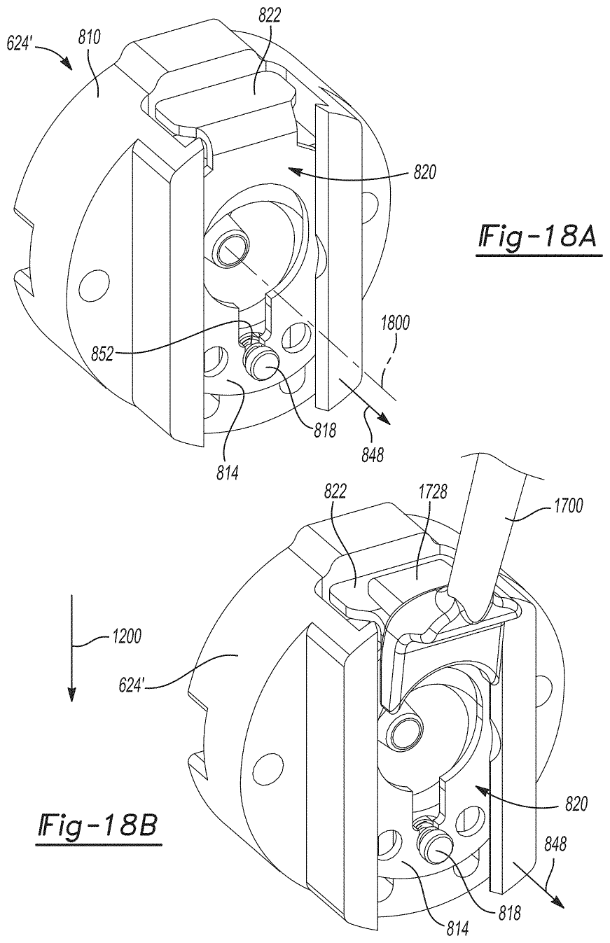

With reference to FIG. 18A, a receiver 624′ according to at least one example embodiment is provided. The receiver 624′ is the same as the receiver 624 of FIG. 6 (and includes the same features having the same reference numerals) except that it is rotated 180° about a central axis 1800 with respect to a bucket (not shown) to which it is attached. The receiver 624′ is shown in a lock state.

Referring to FIG. 18B, tool 1700 may be used to facilitate unlocking of the receiver 624′. The tool 1700 may be moved toward the receiver 624′ until the plate 1730 of the tool 1700 engages the flange 822 of the latch plate 814. The tool 1700 may be moved in the fourth direction 1200 to translate the latch plate 814 in the fourth direction 1200. As the latch plate 814 translates, the pin edge 870 (shown in FIG. 8A) of the latch plate 814 moves out of the second annular groove 852 of the trigger pin 818 such that the trigger pin is forced in the first direction 848 by the first spring 842 (shown in FIG. 8A), thereby retaining the latch plate 814 in the release state.

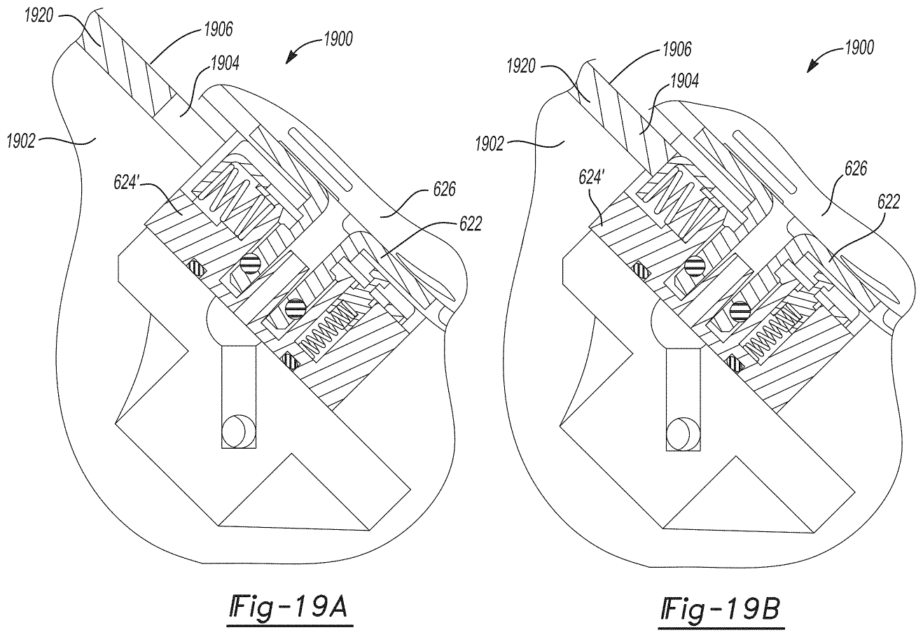

In at least one example embodiment, a separation apparatus may include features to facilitate ease of use of a selective coupling tool, such as a channel. With reference to FIGS. 19A-19B, a separation apparatus 1900 according to at least one example embodiment is provided. The separation apparatus 1900 may be the same as the separation apparatus 600 of FIG. 6, except as otherwise provided below. The separation apparatus includes the receiver 624′.

In at least one example embodiment, the separation apparatus 1900 includes a bucket 1902 that at least partially defines a rod translation channel 1904. The rod translation channel 1904 may include a hole, groove, aperture, or other feature. The rod translation channel 1904 is aligned with a portion of the latch plate 814. The rod translation channel 1904 may be defined in a sidewall 1906 of each bucket 1902, for example, extending from an upper portion of the bucket (not shown) to a point 1908 adjacent the latch plate 814 of the receiver 624′.

A tool or release rod 1920 be movable within the rod translation channel 1904. In the example embodiment shown, the rod translation channel 1904 may be receive at least a portion of the rod 1920 such that the rod 1920 can contact the flange 822 of the latch plate 814. The tool 1920 may be used to release the receiver 624′ as described above in the discussion accompanying FIGS. 18A-18B. Accordingly, the receiver 624′ may be released without requiring the insertion of any tool into the space between the bladder 626 and the sidewall 1906.

In at least one example embodiment, the tool 1920 may remain in the sidewall 1906 and be actuated from a noncontact position, shown in FIG. 19A, to a contact position, shown in FIG. 19B. Additionally or alternatively, the tool 1920 may be selectively received in the rod translation channel 1904 (e.g., during a maintenance operation, etc.) and moved within the rod translation channel 1904 until the end of the tool 1920 contacts the latch plate 814, as shown in FIG. 19B.

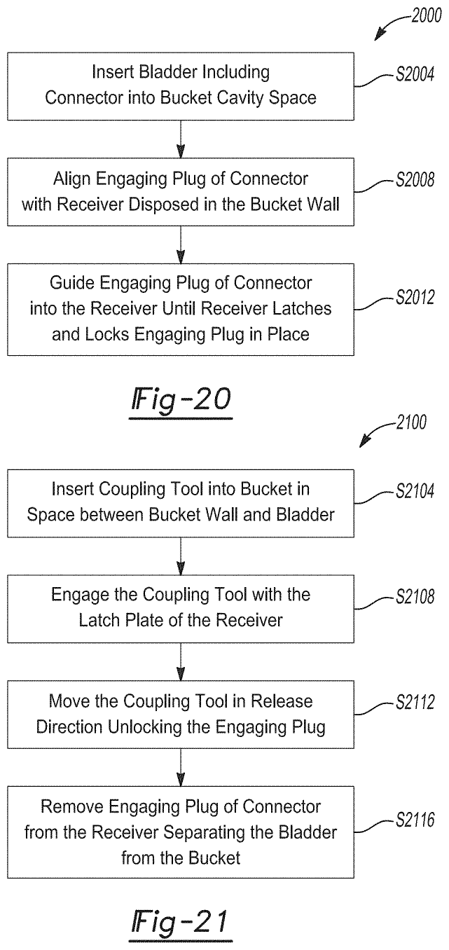

FIG. 20 is a flow diagram of a method 2000 for engaging a bladder with a separation cell of a separation apparatus in accordance with embodiments of the present disclosure. The method 2000 begins at S2004 by inserting the bladder including a connector (e.g., an integrated connector) into the a region between the bladder and a bucket. In at least one example embodiment, S2004 may be performed during an initial setup of the separation apparatus and/or during a maintenance of the separation apparatus. In at least one example embodiment, the bladder may be inserted such that the connector, disposed at a bottom of the bladder, is inserted into the cavity space first and lowered until the connector is proximate a receiver in the bucket.

Next, the method 2000 proceeds at S2008 by aligning an engaging plug of the connector with the receiver of the bucket. In at least one example embodiment, a tool, such as the tool 900 (shown in FIGS. 9A-9D), the tool 1700 (shown in FIGS. 17A-17D), or the tool 1920 (shown in FIGS. 19A-19B) may be used by a technician to aid in the alignment of the engaging plug with the receiver. For instance, a length of a shaft of the tool may be set such that when a coupling end of the tool is inserted into the bucket and moved toward a bottom wall of the bucket, with a handle of the tool disposed adjacent a top of the bucket, a cradle of the tool may be positioned substantially concentrically with a receiving aperture of the receiver. In at least one example embodiment, the shaft of tool may include gradations, marks, or other indicia that allow a technician to determine the relative depth of the coupling end of the tool from a top of the bucket. During S2008, the cradle of the tool may be caused to contact and support a portion of the outer diameter of the engaging plug of the connector. When aligned, an axis of the engaging plug may be substantially colinear with an axis of the receiving aperture (e.g., within about a 3-5 millimeter radius measured at the center from the axis of the receiver aperture, etc.). This step may correspond to the position of the engaging plug shown at least in FIGS. 8A, 10, and 13.

Once aligned with the receiver, the method 2000 may continue at S2012 by guiding the engaging plug of the connector into the receiver (e.g., the receiving aperture) until the receiver latches and locks the engaging plug in place. S2012 may correspond to the engagement of the connector with the receiver as described in conjunction with FIGS. 8A-8C. In at least one example embodiment, the tool may be used to cause a movement of the engaging plug in the direction of the receiver. For instance, the selective coupling tool may be moved, rotated, and/or pivoted at the handle to move the cradle of the coupling, that is in contact with the engaging plug, in a direction toward the receiver. In some examples, the bladder may be pushed against the connector to cause the trigger pin to release the latch plate and lock the engaging plug in place (e.g., as shown at least in FIGS. 8C and 11A). The pushing may be provided by inserting a tool into the bucket in a space behind the bladder and the connector and then manipulating the tool to apply a force against the bladder that engages the connector with the receiver.

FIG. 21 is a flow diagram of a method 2100 for disengaging a bladder from a bucket of a separation apparatus in accordance with at least one embodiment. The method 2100 may be performed during a maintenance operation (e.g., by a technician) or a bladder changeover operation, for example.

The method 2100 may begin at S2104 by inserting the tool, such as the tool 900 (shown in FIGS. 9A-9D), the tool 1700 (shown in FIGS. 17A-17D), or the tool 1920 (shown in FIGS. 19A-19B) into a space between a bladder and a receiver of the bucket. During this step, the bladder is connected to the receiver, such as shown in FIG. 8C. In at least one example embodiment, the tool may be moved in a space between the bladder and the receiver and/or a bucket wall of the bucket until a coupling end of the tool contacts the connector. In at least one example embodiment, the tool may be inserted into the bucket such that release pins are facing the receiver of the bucket.

Next, the method 2100 may proceed at S2108 by engaging the tool with a latch plate of the receiver. In at least one example embodiment, the tool may be moved such that the release pins are inserted, at least partially, into release holes of the latch plate.

At 52112, once the release pins are engaged with the latch plate, the selective coupling tool may be moved in a release direction to unlock an engaging plug from the receiver. The release direction may depend on the orientation of the receiver and latch plate in the bucket.

When the latch plate is moved to the release position, the method 2100 may continue at 52116 by removing the engaging plug of the connector from the receiver, physically separating the bladder from the bucket. Removing the engaging plug may include moving the connector in a direction away from the receiver. Once clear of the receiver, the bladder and integrated connector may be removed from the bucket. If part of a bladder changeover, or maintenance, operation a technician may decide to insert and attach a new bladder, or a repaired bladder, with integrated connector as described in conjunction with the method 2000 of FIG. 20. As can be appreciated, the methods 2000 and 2100 may be repeated for the life of the separation apparatus.

Any of the steps, functions, and operations discussed herein can be performed continuously and automatically.

While the flowcharts have been discussed and illustrated in relation to a particular sequence of events, it should be appreciated that changes, additions, and omissions to this sequence can occur without materially affecting the operation of the disclosed embodiments, configuration, and aspects.

The exemplary systems and methods of this disclosure have been described in relation to couplings between bladders and fluid supply sources. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

A number of variations and modifications of the disclosure can be used. It would be possible to provide for some features of the disclosure without providing others.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

Exemplary aspects are directed to a selective coupling assembly, comprising: a bucket comprising a sidewall extending from an open end of the bucket to a closed end of the bucket and a cavity disposed between the open end of the bucket and the closed end of the bucket; a receiver attached to the sidewall, the receiver comprising: a body comprising a receiver lumen passing from a first side of the body through a second side of the body and a receiving aperture disposed around the receiver lumen; and a latch plate slidably attached to the body, the latch plate comprising an aperture and an aperture axis that is arranged parallel to an axis of the receiver lumen; a bladder comprising a sealed expandable chamber and a fluid flow port disposed in the bladder and passing from an interior volume of the sealed expandable chamber to an outside of the bladder; and a connector attached (or alternatively, affixed) to the bladder and disposed at least partially in the fluid flow port, the connector comprising: a base; an engaging plug protruding from the base; and a connector lumen passing through the base and the engaging plug, the connector lumen providing a fluid flow path from the interior volume of the sealed expandable chamber to the outside of the bladder.

Any one or more of the above aspects include wherein the receiver is at least partially disposed in the sidewall. Any one or more of the above aspects include wherein the engaging plug further comprises: at least one recess offset a first distance from the base of the engaging plug; and a groove arranged around a periphery of the engaging plug, the groove offset a second distance from the base of the engaging plug. Any one or more of the above aspects include wherein the connector further comprises an O-ring disposed at least partially in the groove. Any one or more of the above aspects include wherein the connector is made from a polymer (e.g., a plastic material), and wherein the connector is attached (or alternatively, affixed) to the bladder via at least one weld. Any one or more of the above aspects include wherein the connector is moveable between a lock state with the receiver and an unlocked state with the receiver, wherein, in the lock state, the bladder is fixedly coupled with the bucket, and wherein, in the unlocked state, the bladder is decoupled from the bucket. Any one or more of the above aspects include wherein, in the lock state, the engaging plug is disposed at least partially in the receiving aperture of the receiver and a portion of the latch plate is disposed in the at least one recess of the engaging plug. Any one or more of the above aspects include wherein, in the lock state, a fluid flow path is formed between the interior volume of the sealed expandable chamber and the receiver lumen of the receiver, and wherein the fluid flow path is unimpeded by any valve between the bladder and the receiver. Any one or more of the above aspects include wherein the engaging plug further comprises: at least one recess offset a first distance from the base of the engaging plug; and a compliant portion arranged around a periphery of the engaging plug, the compliant portion corresponding to a seal between the engaging plug and the receiver aperture, the compliant portion offset a second distance from the base of the engaging plug. Any one or more of the above aspects include wherein the compliant portion is an elastically flexible ridge protruding from the engaging plug.

Exemplary aspects are directed to a bladder assembly, comprising: a flexible material comprising a sealed expandable chamber; a fluid flow port disposed passing from an interior volume of the sealed expandable chamber to an outside of the sealed expandable chamber; and a connector attached (or alternatively, affixed) to the flexible material and disposed at least partially in the fluid flow port, the connector comprising: a base; an engaging plug protruding from the base; and a connector lumen passing through the base and the engaging plug, the connector lumen providing a fluid flow path from the interior volume of the sealed expandable chamber to the outside of the flexible material.

Any one or more of the above aspects include wherein the connector is attached (or alternatively, affixed) to the flexible material via a seal surrounding the fluid flow port and joining a portion of the flexible material to the base of the connector.

Exemplary aspects are directed to a method of coupling a bladder to a bucket, comprising: inserting a bladder comprising an integral connector into a cavity of a bucket; aligning the integral connector with a receiver disposed in a sidewall of the bucket; guiding the integral connector into the receiver; and applying a locking force to the integral connector such that a latch of the receiver engages with a portion of the integral connector and prevents axial movement of the integral connector relative to the receiver.

Any one or more of the above aspects include wherein guiding the integral connector comprises: inserting a selective coupling tool into the cavity of the bucket; moving the selective coupling tool into contact with a portion of the integral connector; and manipulating the selective coupling tool causing a movement of the integral connector in a direction of the receiver. Any one or more of the above aspects include wherein moving the selective coupling tool into contact with the portion of the integral connector comprises: positioning the selective coupling tool relative to a top surface of the bucket; and aligning a gradation on a shaft of the selective coupling tool with a reference point at the top surface of the bucket. Any one or more of the above aspects include wherein the bladder comprises: a sealed expandable chamber and a fluid flow port disposed in the bladder and passing from an interior volume of the sealed expandable chamber to an outside of the bladder. Any one or more of the above aspects include wherein the integrated connector comprises: a base; an engaging plug protruding from the base; and a connector lumen passing through the base and the engaging plug, the connector lumen providing a fluid flow path from the interior volume of the sealed expandable chamber to the outside of the bladder.

Exemplary aspects are directed to a method of decoupling a bladder from a bucket, comprising: inserting an end of a selective coupling tool into a cavity of a bucket in a space between the bladder and a sidewall of the bucket; engaging the end of the selective coupling tool with a latch plate of a receiver disposed at least partially in the sidewall of the bucket; moving the coupling tool in a release direction causing the latch plate to translate from a lock state, where a portion of the latch plate is engaged with a portion of a connector of the bladder, to a release state, where the portion of the latch plate is disengaged with the portion of the connector of the bladder; and moving the connector of the bladder in a direction away from the receiver causing the bladder to separate from the bucket.

Any one or more of the above aspects include wherein engaging the end of the selective coupling tool with the latch plate of the receiver comprises: aligning a pin disposed in the end of the selective coupling tool with a corresponding hole disposed in the latch plate; and inserting a portion of the pin into the corresponding hole disposed in the latch plate. Any one or more of the above aspects include wherein aligning the pin disposed in the end of the selective coupling tool with the corresponding hole disposed in the latch plate comprises: positioning the selective coupling tool relative to a top surface of the bucket; and aligning a gradation on a shaft of the selective coupling tool with a reference point at the top surface of the bucket.

Exemplary aspects are directed to a selective coupling tool, comprising: a shaft extending from a proximal end to a distal end; and a forked extension protruding from the distal end, the forked protrusion having a first side and a second side disposed opposite the first side, wherein the forked extension comprises: a cradle comprising a contact surface running from the first side to the second side; and a release pin protruding from the second side.

Any one or more of the above aspects include wherein the release pin comprises a frustoconical protrusion. Any one or more of the above aspects include a handle connected to the proximal end of the shaft; and at least one gradation disposed along a length of the shaft. Any one or more of the above aspects include wherein the at least one gradation is etched into a portion of the shaft and wrapping around at least a portion of an outer surface of the shaft.

Exemplary aspects are directed to a blood separation apparatus, comprising: a rotor; a bucket attached to the rotor, the bucket comprising a sidewall extending from an open end of the bucket to a closed end of the bucket and a cavity disposed between the open end of the bucket and the closed end of the bucket; a receiver attached to the sidewall, the receiver comprising: a body comprising a receiver lumen passing from a first side of the body through a second side of the body and a receiving aperture disposed around the receiver lumen; and a latch plate slidably attached to the body, the latch plate comprising an aperture and an aperture axis that is arranged parallel to an axis of the receiver lumen; a bladder comprising a sealed expandable chamber and a fluid flow port disposed in the bladder and passing from an interior volume of the sealed expandable chamber to an outside of the bladder; and a connector attached (or alternatively, affixed) to the bladder and disposed at least partially in the fluid flow port, the connector comprising: a base; an engaging plug protruding from the base; and a connector lumen passing through the base and the engaging plug, the connector lumen providing a fluid flow path from the interior volume of the sealed expandable chamber to the outside of the bladder.

Any one or more of the above aspects include wherein the base is substantially flat comprising a planar substrate.

Exemplary aspects are directed to a selective coupling assembly, comprising: a receiver disposed at least partially within a sidewall of a bladder holder, the receiver comprising: a body comprising a receiver lumen passing from a first side of the body through a second side of the body and a receiving aperture disposed around the receiver lumen; and a latch plate slidably attached to the body, the latch plate comprising an aperture and an aperture axis that is arranged parallel to an axis of the receiver lumen; a bladder comprising an expandable chamber and a fluid flow port disposed in the bladder and passing from an interior volume of the expandable chamber to an outside of the bladder; and a connector attached (or alternatively, affixed) to the bladder and disposed at least partially in the fluid flow port, the connector comprising: a base; an engaging plug protruding from the base; and a connector lumen passing through the base and the engaging plug, the connector lumen providing a fluid flow path from the interior volume of the expandable chamber to the outside of the bladder.

Any one or more of the above aspects include wherein the bladder is moveable from a separated position disposing the bladder outside of a receiving volume of the bladder holder to a holding position disposing the bladder inside the receiving volume of the bladder holder, and wherein the receiver is at least partially disposed within the receiving volume of the bladder holder. Any one or more of the above aspects include wherein the connector is moveable between a lock state with the receiver and an unlocked state with the receiver, wherein, in the lock state, the bladder is fixedly coupled with the bladder holder, wherein, in the unlocked state, the bladder is decoupled from the bladder holder, and wherein the connector is moveable between the lock state and the unlocked state from a space inside the receiving volume of the bladder holder. Any one or more of the above aspects include wherein the connector is moveable between the lock state and the unlocked state without use of a tool. Any one or more of the above aspects include wherein the connector is moveable between the lock state and the unlocked state by inserting a tool from the outside of the receiving volume of the bladder holder into a space inside of the receiving volume of the bladder holder. Any one or more of the above aspects include wherein the bladder holder is a bucket of a separation apparatus, wherein the sidewall extends from an open end of the bucket to a closed end of the bucket, and wherein the receiving volume is disposed between the open end of the bucket and the closed end of the bucket.

Exemplary aspects are directed to an interconnection assembly, comprising: a receiver, comprising: a body comprising a receiver lumen passing from a first side of the body through a second side of the body and a receiving aperture disposed around the receiver lumen; and a latch plate slidably attached to the body, the latch plate comprising an aperture and an aperture axis that is arranged parallel to an axis of the receiver lumen; and a connector, comprising: a base; an engaging plug protruding from the base; and a connector lumen passing through the base and the engaging plug.

Any one or more of the above aspects further comprising: a bladder comprising an expandable chamber and a fluid flow port disposed in the bladder and passing from an interior volume of the expandable chamber to an outside of the bladder, wherein the connector is operatively attached to the fluid flow port such that the connector lumen provides a fluid flow path from the interior volume of the expandable chamber to the outside of the bladder.

Any one or more of the above aspects/embodiments as substantially disclosed herein.

Any one or more of the aspects/embodiments as substantially disclosed herein optionally in combination with any one or more other aspects/embodiments as substantially disclosed herein.

One or means adapted to perform any one or more of the above aspects/embodiments as substantially disclosed herein.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A selective coupling assembly comprising:
a bucket including,
a wall extending from an open end of the bucket to a closed end of the bucket, the wall at least partially defining a cavity between the open end and the closed end, and
a receiver attached to the wall, the receiver including,
a body defining a receptacle and a receiver lumen extending between a first side of the body and a second side of the body, the receiver lumen having a lumen axis, and
a latch plate slidably attached to the body, the latch plate defining an aperture and having an aperture axis parallel the lumen axis;
a bladder defining a sealed expandable chamber and a fluid flow port passing from an interior volume of the sealed expandable chamber to an outside of the bladder; and
a connector attached to the bladder and at least partially in the fluid flow port, the connector including,
a base, and
a plug protruding from the base, the connector defining a connector lumen in the base and the plug, the connector lumen providing a fluid flow path from the interior volume of the sealed expandable chamber to an outside of the bladder.

2. The selective coupling assembly of claim 1, wherein the receiver is at least partially in the wall.

3. The selective coupling assembly of claim 2, wherein the plug further includes,
a recess offset a first distance from the base of the plug, and
a groove defined in a periphery of the plug, the groove offset a second distance from the base of the plug.

4. The selective coupling assembly of claim 3, wherein the connector further includes an O-ring at least partially in the groove.

5. The selective coupling assembly of claim 4, wherein
the connector comprises a plastic material, and
the connector is attached to the bladder via at least one weld.

6. The selective coupling assembly of claim 5, wherein
the connector is configured to move between a lock state with the receiver and an unlocked state with the receiver,
in the lock state, the bladder is coupled to the bucket, and
in the unlocked state, the bladder is decoupled from the bucket.

7. The selective coupling assembly of claim 6, wherein, in the lock state, the plug is disposed at least partially in the receptacle and a portion of the latch plate is in the recess of the plug.

8. The selective coupling assembly of claim 7, wherein
in the lock state, a fluid flow path is between the interior volume of the sealed expandable chamber and the receiver lumen of the receiver, and
the fluid flow path is unimpeded by any valve between the bladder and the receiver.

9. A bladder assembly comprising:
a flexible material defining a sealed expandable chamber, the flexible material defining a fluid flow port passing from an interior volume of the sealed expandable chamber to an outside of the sealed expandable chamber; and
a connector attached to the flexible material and at least partially in the fluid flow port, the connector including,
a base, and
a plug protruding from the base, the connector defining a connector lumen through the base and the plug, the connector lumen providing a fluid flow path from the interior volume of the sealed expandable chamber to an outside of the flexible material.

10. The bladder assembly of claim 9, wherein the connector is attached to the flexible material via a seal surrounding the fluid flow port and joining a portion of the flexible material to the base of the connector.

11. A method of coupling a bladder to a bucket, the method comprising:
inserting a bladder including an integral connector into a cavity of a bucket;
aligning the integral connector with a receiver in a wall of the bucket;
guiding the integral connector into the receiver; and
applying a force to the integral connector to engage a latch of the receiver with a portion of the integral connector and reduce or prevent axial movement of the integral connector relative to the receiver.

12. The method of claim 11, wherein the guiding includes,
inserting a selective coupling tool into the cavity of the bucket,
moving the selective coupling tool into contact with a portion of the integral connector, and
manipulating the selective coupling tool to cause a movement of the integral connector in a direction of the receiver.

13. The method of claim 12, wherein the moving includes,
positioning the selective coupling tool relative to a top surface of the bucket, and
aligning an indicium on a shaft of the selective coupling tool with a reference point at the top surface of the bucket.

14. The method of claim 13, wherein the bladder defines a sealed expandable chamber and a fluid flow port passing from an interior volume of the sealed expandable chamber to an outside of the bladder.

15. The method of claim 14, wherein the integrated connector includes,
a base, and
a plug protruding from the base, a connector lumen passing through the base and the plug, the connector lumen providing a fluid flow path from the interior volume of the sealed expandable chamber to an outside of the bladder.

16. A method of decoupling a bladder from a bucket, the method comprising:
inserting a selective coupling tool at least partially into a cavity of a bucket in a region between the bladder and a wall of the bucket;
engaging the selective coupling tool with a latch plate of a receiver at least partially in the wall of the bucket;
moving the selective coupling tool to cause the latch plate to translate from a lock state, to a release state, in the lock state, a portion of the latch plate is engaged with a portion of a connector of the bladder, in the release state, the portion of the latch plate is disengaged with the portion of the connector of the bladder; and
moving the connector away from the receiver to cause the bladder to separate from the bucket.

17. The method of claim 16, wherein the engaging includes,
aligning a pin of the selective coupling tool with a corresponding hole defined in the latch plate; and
inserting at least a portion of the pin into the corresponding hole.

18. The method of claim 17, wherein the aligning includes aligning an indicium on a shaft of the selective coupling tool with a reference point at a top surface of the bucket.

19. The method of claim 16, wherein the engaging includes contacting the selective coupling tool with a flange of the latch plate.

20. A selective coupling tool comprising:
a shaft extending from a proximal end to a distal end; and
a forked extension protruding from the distal end, the forked extension having a first side and a second side opposite the first side, the forked extension including,
a cradle including a contact surface between the first side and the second side, and
a protrusion on the second side.

21. The selective coupling tool of claim 20, wherein the protrusion is a frustoconical protrusion.

22. The selective coupling tool of claim 20, wherein the protrusion is a plate.

23. The selective coupling tool of claim 20, further comprising:
a handle connected to the proximal end of the shaft; and
an indicium the shaft.

24. The selective coupling tool of claim 23, wherein the indicium is etched into a portion of the shaft and extends around at least a portion of an outer surface of the shaft.

25. A blood separation apparatus comprising:
a rotor;
a bucket attached to the rotor, the bucket including,
a wall extending from an open end of the bucket to a closed end of the bucket, the wall defining a cavity between the open end and the closed end;
a receiver attached to the wall, the receiver including,
a body defining a receptacle and a receiver lumen extending between a first side of the body and a second side of the body, the receiver lumen having a lumen axis, and
a latch plate slidably attached to the body, the latch plate defining an aperture and having an aperture axis parallel to the lumen axis;
a bladder defining a sealed expandable chamber and a fluid flow port extending between an interior volume of the sealed expandable chamber to an outside of the bladder; and
a connector attached to the bladder, the connector at least partially in the fluid flow port, the connector including,
a base,
a plug protruding from the base, and
a connector lumen through the base and the plug, the connector lumen providing a fluid flow path from the interior volume of the sealed expandable chamber to an outside of the bladder.

26. A selective coupling assembly comprising:
a receiver at least partially within a wall of a bladder holder, the receiver including,
a body defining a receptacle and a first lumen extending from a first side of the body to a second side of the body, the first lumen having a lumen axis, and
a latch plate slidably attached to the body, the latch plate defining an aperture and having an aperture axis parallel to the lumen axis;
a bladder defining an expandable chamber and a fluid flow port passing from an interior volume of the expandable chamber to an outside of the bladder; and
a connector attached to the bladder, the connector at least partially in the fluid flow port, the connector including,
a base,
a plug protruding from the base, the connector defining a connector lumen through the base and the plug, the connector lumen providing a fluid flow path from the interior volume of the expandable chamber to an outside of the bladder.

27. The selective coupling assembly of claim 26, wherein
the bladder is configured to be moved from a first position outside of the bladder holder to a second position inside the bladder holder, and
the receiver is at least partially within the bladder holder.

28. The selective coupling assembly of claim 27, wherein
the connector is configured to be moved between a lock state with the receiver and an unlocked state with the receiver,
in the lock state, the bladder is coupled to the bladder holder,
in the unlocked state, the bladder is decoupled from the bladder holder, and
the connector is configured to be moved between the lock state and the unlocked state from a region inside the bladder holder.

29. The selective coupling assembly of claim 28, wherein the connector is configured to be moved between the lock state and the unlocked state without use of a tool.

30. The selective coupling assembly of claim 28, wherein the connector is configured to be moved between the lock state and the unlocked state by inserting a tool from an outside of the bladder holder into the region.

31. The selective coupling assembly of claim 28, wherein
the bladder holder is a bucket of a separation apparatus, and
the wall extends from an open end of the bucket to a closed end of the bucket.

32. An interconnection assembly comprising:
a receiver including,
a body defining a receptacle and a receiver lumen extending between a first side of the body to second side of the body, the receiver lumen having a lumen axis, and
a latch plate slidably attached to the body, the latch plate defining an aperture having an aperture axis parallel to the lumen axis; and
a connector including,
a base,
a plug protruding from the base, the connector defining a connector lumen through the base and the plug.

33. The interconnection assembly of claim 32, further comprising:
a bladder defining an expandable chamber and a fluid flow port passing from an interior volume of the expandable chamber to an outside of the bladder, wherein the connector is operatively attached to the fluid flow port such that the connector lumen provides a fluid flow path from the interior volume of the expandable chamber to an outside of the bladder.

34. The interconnection assembly of claim 32, wherein the plug further includes,
a recess offset a first distance from the base of the plug, and
a compliant portion arranged around a periphery of the plug, the compliant portion configured to create a seal between the plug and the receptacle, the compliant portion offset a second distance from the base.

35. The interconnection assembly of claim 34, wherein the compliant portion is an elastically flexible ridge protruding from the plug.
